(19) United States
(12) Patent Application Publication
(10) Pub. No.: US 2024/0335466 A1
(43) Pub. Date: Oct. 10, 2024

(54) NICOTINAMIDE MONONUCLEOTIDE DERIVATIVES FOR USE IN THE TREATMENT OF SAPHO SYNDROME

(71) Applicant: NUVAMID SA, Epalinges (CH)

(72) Inventors: Laurent GARÇON, Epalinges (CH); Guillaume BERMOND, Epalinges (CH); Cécile CROS, Epalinges (CH); Matthias CANAULT, Epalinges (CH)

(73) Assignee: NUVAMID SA, Epalinges (CH)

(21) Appl. No.: 18/292,620

(22) PCT Filed: Aug. 2, 2022
(86) PCT No.: PCT/EP2022/071742

§ 371 Date: Jan. 26, 2024

(30) Foreign Application Priority Data
Aug. 2, 2021 (EP) 21189185.8

(51) Int. Cl. A61K 31/7084; A61K 31/706; A61K 45/06; A61P 19/00; A61P 19/02

(52) U.S. Cl. CPC A61K 31/7084; A61K 31/706; A61K 45/06; A61P 19/00; A61P 19/02

(57) ABSTRACT

Nicotinamide mononucleotide derivatives of Formula (I)

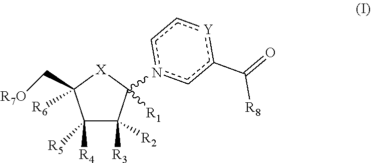

or pharmaceutically acceptable salts or solvates thereof, for use in the treatment of SAPHO syndrome in a subject in need thereof. The subject suffers from one first symptom which is an osteoarticular symptom of SAPHO syndrome selected from synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes, and at least one second symptom of SAPHO syndrome different from the first system.

### FIELD OF INVENTION

The present invention relates to the treatment of SAPHO syndrome. In particular, the present invention relates to the use of nicotinamide mononucleotide derivatives, in the treatment of SAPHO syndrome, in a subject in need thereof.

### BACKGROUND OF INVENTION

The SAPHO syndrome is a rare disease which was first described in 1987, with a prevalence estimated to be less than 1 to 10,000. It encompasses several chronic conditions that affect bones, joints and/or skin.

The SAPHO syndrome was given its acronymic name based upon the main manifestations usually observed during the disease: synovitis, acne, pustulosis, hyperostosis, and osteitis. There is a wide variability in these osteoarticular and cutaneous manifestations of the SAPHO syndrome.

Bone and joint (i.e. “osteoarticular”) manifestations of SAPHO syndrome are the hallmarks of the disorder and occur regardless of the presence of active dermatologic findings. Osteoarticular manifestations include synovitis, osteitis, hyperostosis, as well as changes consistent with axial spondyloarthritis, affecting the sacroiliac joints and spine, enthesitis and diffuse idiopathic skeletal hyperostosis (DISH)-like non-marginal enthesophytes. Bone and joint involvement may affect a variety of regions, especially the anterior chest wall, other parts of the axial skeleton, including the sacroiliac joint and spine; and medium to large lower-extremity joints.

More than 60 percent of patients diagnosed with SAPHO develop an associated cutaneous manifestation. Skin findings in patients with SAPHO syndrome include a variety of acneiform and neutrophilic dermatoses. Particularly, palmoplantar pustulosis was reported as the most common cutaneous manifestation, affecting up to 60 percent of patients who develop skin manifestations. Moderate to severe forms of nodulocystic acne, involving the face, chest, and back, and often with residual scarring, are also typical. In addition to acne, other features of follicular occlusion syndromes may be present, including hidradenitis suppurativa.

Additional manifestations may also be present, such as systemic features including fatigue and fever, inflammatory bowel disease (IBD), Crohn disease, and other less frequent manifestations including venous thrombosis, hypertrophic pachymeningitis, uveitis, sciatica, AA amyloidosis with related renal involvement and pleural abnormalities including both parenchymal and pleural changes.

Patients with SAPHO syndrome do not necessarily have all of the above manifestations at the same time, nor successively. Almost all the combinations of symptoms are possible.

Individuals between the ages of 30 and 50 seem most frequently affected, but children and young adults have also been reported, as have older adults. In children, the syndrome is usually referred to as chronic recurrent multifocal osteomyelitis (CRMO). There is an apparent female predominance, particularly among patients less than 30 years of age at onset of the syndrome.

The pathogenesis of SAPHO syndrome is not well understood and a number of genetic and environmental (eg, infectious) factors and immune dysregulation have been proposed to contribute to disease susceptibility and development.

The diagnosis of SAPHO syndrome is difficult, given the wide variety of clinical manifestations that may occur in the patients. Usually, at least the two following situations lead to diagnose SAPHO syndrome: (1) the case of sterile bone inflammation (which is not caused by bacteria) affecting the thorax, spine or pelvis, with or without skin lesions; and (2) the case of joint damage associated with a skin disease (severe acne, psoriasis or palmoplantar pustulosis, etc.). The diagnosis of SAPHO syndrome remains largely a clinical diagnosis that is based upon the presence of a combination of features. It is also a diagnosis of exclusion that requires an absence of evidence to support an infectious, malignant, or other etiology. Classic rheumatic diseases, such as rheumatoid arthritis, axial spondyloarthritis, or psoriatic arthritis should also be excluded.

SAPHO syndrome is a chronic condition in the vast majority of cases and follows a stable chronic or relapsing-remitting course. Although the course is variable, disabling complications are not common, although peripheral arthritis may be erosive, and bone and limb length discrepancies may occur. Nevertheless, daily life may be disrupted in periods of crisis since bone and joint pain can be very disabling. Skin manifestations can also be very troublesome, in particular for aesthetic reasons. Further, pustules under the feet can be painful and interfere with walking.

The treatment of osteoarticular and cutaneous manifestations of SAPHO syndrome is thus recommended for all patients, not only for symptom relief but also because it is expected to minimize risk for joint injury and further complications. Currently, there is no specific treatment for SAPHO syndrome, which treatment remains mainly symptomatic. Nonsteroidal anti-inflammatory drugs (NSAIDs) are usually prescribed. However, they are often insufficiently effective, which is why other treatments are usually combined with them, such as sulfasalazine or methotrexate. Bisphosphonates (especially pamidronate), have also shown a favorable action in certain cases not only on pain, but also on pustulosis lesions. Anti-TNF-alpha biotherapies may also be an interesting alternative. Prolonged antibiotic treatment is also sometimes considered. In some cases, corticosteroids (such as prednisone) are prescribed, usually in low doses and for a short time, mainly to help with bone and joint pain. Morphine-type analgesics given very occasionally can also bring relief to patients. The cutaneous manifestations need to be managed by a dermatologist. For example, acne is usually sensitive to treatments with retinoids, which however can cause several side-effects. Doxycycline may also be effective against acne, and sometimes seems to relieve bone and joint pain.

Therefore, there is currently no optimal approach for the treatment of SAPHO syndrome. There is thus still a need to provide a treatment for patients suffering from SAPHO syndrome, especially for patients suffering from at least two symptoms of SAPHO syndrome.

The present invention relates to the use of nicotinamide mononucleotide derivatives, in the treatment of SAPHO syndrome. As evidenced in the example part, the nicotinamide mononucleotide derivatives of the invention, in particular nicotinamide mononucleotide (NMN), are able to improve the symptoms of patients suffering from SAPHO syndrome, especially to improve concomitantly at least two symptoms in patients suffering from SAPHO syndrome.

### SUMMARY

This invention thus relates to a compound of Formula (I),

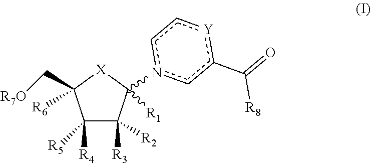

or a pharmaceutically acceptable salt or solvate thereof; wherein R₁-R₈, X and Y are as defined hereafter, for use in the treatment of SAPHO syndrome in a subject in need thereof.

Especially, the subject suffers from:
one first symptom which is an osteoarticular symptom of SAPHO syndrome selected from synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes;

and
at least one second symptom of SAPHO syndrome, different from the first symptom, selected from:
   osteoarticular symptoms selected from synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes;
   cutaneous symptoms selected from palmoplantar pustulosis, acne, hidradenitis suppurativa, and pustular psoriasis; and
   symptoms selected from fatigue, fever, inflammatory bowel disease (IBD), Crohn disease, venous thrombosis, hypertrophic pachymeningitis, uveitis, sciatica, AA amyloidosis with related renal involvement, pleural abnormalities with parenchymal changes and pleural abnormalities with pleural changes.

In one embodiment, the patient does not suffer from psoriatic arthritis.

In one embodiment, X represents an oxygen. In one embodiment, R₁ and R₆ are identical and represent hydrogen. In one embodiment, R₃ and R₄ are identical and represent hydrogen. In one embodiment, R₂ and R₅ are identical and represent OH. In one embodiment, Y is selected from CH or CH₂. In one embodiment, R₈ is NH₂.

In one embodiment, R₇ is selected from H, P(O)R₉R₁₀ or

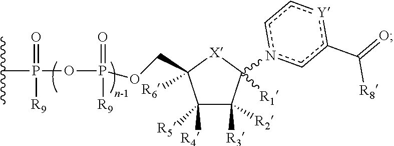

wherein R₉ and R₁₀ as well as R_{1′}-R_{8′}, X′ and Y′ are as described hereafter.

In one embodiment, the compound of Formula (I) is selected from compounds 001 to 014 and pharmaceutically acceptable salts and solvates thereof.

In one embodiment, the subject suffers from two or more symptoms of SAPHO syndrome selected from synovitis, osteitis, primitive inflammatory osteitis, hyperostosis, axial spondyloarthritis, arthritis, enthesitis, diffuse idiopathic skeletal hyperostosis (DISH)-like non-marginal enthesophytes, palmoplantar pustulosis, acne, hidradenitis suppurativa, and pustular psoriasis.

In one embodiment, the subject suffers from:
one or more osteoarticular symptoms of SAPHO syndrome selected from synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes; and
one or more cutaneous symptoms of SAPHO syndrome selected from palmoplantar pustulosis, acne, hidradenitis suppurativa, and pustular psoriasis.

In one embodiment, at least two symptoms of SAPHO syndrome are treated. In one embodiment, the at least two treated symptoms are selected from synovitis, osteitis, primitive inflammatory osteitis, hyperostosis, axial spondyloarthritis, arthritis, enthesitis, diffuse idiopathic skeletal hyperostosis (DISH)-like non-marginal enthesophytes, palmoplantar pustulosis, acne, hidradenitis suppurativa, and pustular psoriasis.

In one embodiment, the compound of Formula (I) is to be administered simultaneously, separately or sequentially with at least one further pharmaceutically active agent selected from naproxen, ibuprofen, indomethacin, diclofenac, celecoxib, etoricoxib, mefenamic acid, high dose aspirin, doxycycline, minocycline, trimethoprim, sulfamethoxazole, azithromycin, clindamycin, methotrexate, etanercept, adalimumab, infliximab, certolizumab pegol, golimumab, tocilizumab, sarilumab, siltuximab, olokizumab, elsilimomab, clazakizumab, sirukumab, levilimab, acitretin, isotretinoin, retinol, retinoic acid, adapalene, alitretinoin, bexarotene, adapalene, pamidronate, risedronate, alendronate, ibandronate, zoledronic acid, etidronate, sulfasalazine, sulfomethoxasol, sulfisoxasol, colchicine, prednisone, hydrocortisone, prednisolone, dexamethasone, methylprednisolone, triamcinolone, betamethasone, oxycodone, hydrocodone, codeine, fentanyl, hydromorphone, oxymorphone, caltonin.

In one embodiment, the compound of Formula (I) is to be administered simultaneously, separately or sequentially with at least one further food supplement or plant extract selected from S-adenosylmethionine, Boswellic acids, capsaicin or Capsicum frutescens, curcumin, turmeric, avocado soybean unsaponifiables, Uncaria tomentosa, fish oil, omega 3 fatty acids (EPA and DHA), gamma linoleic acid, ginger, Zingiber officinale, cannabidiol, CBD, chondroitin, glucosamine, and harpagophytum.

The invention further relates to a pharmaceutical composition for use in the treatment of SAPHO syndrome in a subject in need thereof, comprising at least one compound of formula (I) as herein defined and at least one pharmaceutically acceptable carrier.

### Definitions

The definitions and explanations below are for the terms as used throughout the entire application, including both the specification and the claims.

When describing the compounds of the invention, the terms used are to be construed in accordance with the following definitions, unless indicated otherwise.

Unless indicated otherwise, the nomenclature of substituents that are not explicitly defined herein are arrived at by naming the adjacent functionality toward the point of attachment followed by the terminal portion of the functionality. For example, the substituent “arylalkyl” refers to the group -(aryl)-(alkyl).

In the present invention, the following terms have the following meanings:

The term “alkyl” by itself or as part of another substituent refers to a hydrocarbyl radical of Formula CₙH₂ₙ₊₁ wherein n is a number greater than or equal to 1. Generally, alkyl groups of this invention comprise from 1 to 12 carbon atoms, preferably from 1 to 8 carbon atoms, more preferably from 1 to 6 carbon atoms, still more preferably 1 or 2 carbon atoms. Alkyl groups may be linear or branched. Suitable alkyl groups include methyl, ethyl, n-propyl, i-propyl, n-butyl, i-butyl, s-butyl and t-butyl, pentyl and its isomers (e.g. n-pentyl, iso-pentyl), hexyl and its isomers (e.g. n-hexyl, isohexyl), heptyl and its isomers (e.g. n-heptyl, iso-heptyl), octyl and its isomers (e.g. n-octyl, iso-octyl), nonyl and its isomers (e.g. n-nonyl, iso-nonyl), decyl and its isomers (e.g. n-decyl, iso-decyl), undecyl and its isomers, dodecyl and its isomers. Preferred alkyl groups include methyl, ethyl, n-propyl, i-propyl, n-butyl, i-butyl, s-butyl, t-butyl, n-pentyl, n-hexyl, n-heptyl, n-octyl, n-nonyl and n-decyl. Saturated branched alkyls include, without being limited to, i-propyl, s-butyl, i-butyl, t-butyl, i-pentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl.

Cₓ-C_{y}-alkyl refers to alkyl groups which comprise x to y carbon atoms.

The term “alkenyl” as used herein refers to an unsaturated hydrocarbyl group, which may be linear or branched, comprising one or more carbon-carbon double bonds. Suitable alkenyl groups comprise between 2 and 12 carbon atoms, preferably between 2 and 8 carbon atoms, still more preferably between 2 and 6 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and the like.

The term “alkynyl” as used herein refers to a class of monovalent unsaturated hydrocarbyl groups, wherein the unsaturation arises from the presence of one or more carbon-carbon triple bonds. Alkynyl groups typically, and preferably, have the same number of carbon atoms as described above in relation to alkenyl groups. Non limiting examples of alkynyl groups are ethynyl, 2-propynyl, 2-butynyl, 3-butynyl, 2-pentynyl and its isomers, 2-hexynyl and its isomers and the like.

The term “alkoxy” as used herein refers to any group —O-alkyl, wherein alkyl is as defined above. Suitable alkoxy groups include for example methoxy, ethoxy, n-propoxy, isopropoxy, n-butoxy, t-butoxy, sec-butoxy, and n-pentoxy.

The term “amino acid” as used herein refers to an alpha-aminated carboxylic acid, i.e. a molecule comprising a carboxylic acid functional group and an amine functional group in alpha position of the carboxylic acid group, for example a proteinogenic amino acid or a non-proteinogenic amino acid.

The term “aryl” as used herein refers to a polyunsaturated, aromatic hydrocarbyl group having a single ring (i.e. phenyl) or multiple aromatic rings fused together (e.g. naphthyl) or linked covalently, typically containing 5 to 12 atoms; preferably 6 to 10, wherein at least one ring is aromatic. The aromatic ring may optionally include one to two additional rings (either cycloalkyl, heterocyclyl or heteroaryl) fused thereto. Aryl is also intended to include the partially hydrogenated derivatives of the carbocyclic systems enumerated herein. Non-limiting examples of aryl comprise phenyl, biphenyl, biphenylenyl, 5- or 6-tetralinyl, naphthalen-1- or -2-yl, 4-, 5-, 6- or 7-indenyl, 1-, 2-, 3-, 4- or 5-acenaphthylenyl, 3-, 4- or 5-acenaphthenyl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, 1-, 2-, 3-, 4- or 5-pyrenyl.

The term “cycloalkyl” as used herein is a cyclic alkyl, alkenyl or alkynyl group, that is to say, a monovalent, saturated, or unsaturated hydrocarbyl group having 1 or 2 cyclic structures. Cycloalkyl includes monocyclic or bicyclic hydrocarbyl groups. Cycloalkyl groups may comprise 3 or more carbon atoms in the ring and generally, according to this invention, comprise from 3 to 10, more preferably from 3 to 8 carbon atoms, still more preferably from 3 to 6 carbon atoms. Examples of cycloalkyl groups include but are not limited to cyclopropyl, cyclobutyl, cyclopentyl, cyclohexyl, with cyclopropyl being particularly preferred.

The term “halo” or “halogen” means fluoro, chloro, bromo, or iodo. Preferred halo groups are fluoro and chloro.

The term “haloalkyl”, alone or as part of another group, refers to an alkyl radical having the meaning as defined above wherein one or more hydrogen atoms are replaced with a halogen as defined above. Non-limiting examples of such haloalkyl radicals include chloromethyl, 1-bromoethyl, fluoromethyl, difluoromethyl, trifluoro methyl, 1,1,1-trifluoroethyl and the like. Cₓ-C_{y}-haloalkyl are haloalkyl groups which comprise x to y carbon atoms. Preferred haloalkyl groups are difluoromethyl and trifluoromethyl.

The term “heteroalkyl” means an alkyl group as defined above in which one or more carbon atoms are replaced by a heteroatom selected from oxygen, nitrogen and sulfur atoms. In heteroalkyl groups, the heteroatoms are linked along the alkyl chain only to carbon atoms, i.e. each heteroatom is separated from any other heteroatom by at least one carbon atom. However, the nitrogen and sulphur heteroatoms may optionally be oxidized and the nitrogen heteroatoms may optionally be quaternized. A heteroalkyl is bonded to another group or molecule only through a carbon atom, i.e. the bonding atom is not selected from the heteroatoms included in the heteroalkyl group.

Where at least one carbon atom in an aryl group is replaced with a heteroatom, the resultant ring is referred to herein as a “heteroaryl ring”.

The term “heteroaryl” as used herein by itself or as part of another group refers but is not limited to 5 to 12 carbon-atom aromatic rings or ring systems containing 1 to 2 rings which are fused together or linked covalently, typically containing 5 to 6 atoms; at least one of which is aromatic, in which one or more carbon atoms in one or more of these rings is replaced by oxygen, nitrogen and/or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. Non-limiting examples of such heteroaryl, include: furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b] [1,3] thiazolyl, thieno [3,2-b] furanyl, thieno [3,2-b] thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl.

Where at least one carbon atom in a cycloalkyl group is replaced with a heteroatom, the resultant ring is referred to herein as “heterocycloalkyl” or “heterocyclyl”.

The terms “heterocyclyl”, “heterocycloalkyl” or “heterocyclo” as used herein by itself or as part of another group refer to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen, oxygen and/or sulfur atoms, where the nitrogen and sulfur heteroatoms may optionally be oxidized and the nitrogen heteroatoms may optionally be quaternized. Any of the carbon atoms of the heterocyclic group may be substituted by oxo (for example piperidone, pyrrolidinone). The heterocyclic group may be attached at any heteroatom or carbon atom of the ring or ring system, where valence allows. The rings of multi-ring heterocycles may be fused, bridged and/or joined through one or more spiro atoms. Non limiting exemplary heterocyclic groups include oxetanyl, piperidinyl, azetidinyl, 2-imidazolinyl, pyrazolidinyl imidazolidinyl, isoxazolinyl, oxazolidinyl, isoxazolidinyl, thiazolidinyl, isothiazolidinyl, piperidinyl, 3H-indolyl, indolinyl, isoindolinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, tetrahydropyranyl, tetrahydrofuranyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulf oxide, thiomorpholin-4-ylsulfone, 1,3-dioxolanyl, 1,4-oxathianyl, 1H-pyrrolizinyl, tetrahydro-1,1-dioxothiophenyl, N-formylpiperazinyl, and morpholin-4-yl.

The term “hydroxyalkyl” refers to an alkyl radical having the meaning as defined above wherein one or more hydrogen atoms are replaced with —OH moieties.

The term “thio-alkyl” refers to an alkyl radical having the meaning as defined above wherein one or more hydrogen atoms are replaced with —SH moieties.

The term “non-proteinogenic amino acid” as used herein refers to an amino acid not naturally encoded or found in the genetic code of living organism. Non limiting examples of non-proteinogenic amino acid are ornithine, citrulline, argininosuccinate, homoserine, homocysteine, cysteine-sulfinic acid, 2-aminomuconic acid, δ-aminolevulinic acid, β-alanine, cystathionine, γ-aminobutyrate, DOPA, 5-hydroxytryptophan, D-serine, ibotenic acid, α-aminobutyrate, 2-aminoisobutyrate, D-leucine, D-valine, D-alanine or D-glutamate.

The term “proteinogenic amino acid” as used herein refers to an amino acid that is incorporated into proteins during translation of messenger RNA by ribosomes in living organisms, i.e. Alanine (ALA), Arginine (ARG), Asparagine (ASN), Aspartate (ASP), Cysteine (CYS), Glutamate (glutamic acid) (GLU), Glutamine (GLN), Glycine (GLY), Histidine (HIS), Isoleucine (ILE), Leucine (LEU), Lysine (LYS), Methionine (MET), Phenylalanine (PHE), Proline (PRO), Pyrrolysine (PYL), Selenocysteine (SEL), Serine (SER), Threonine (THR), Tryptophan (TRP), Tyrosine (TYR) or Valine (VAL).

The term “prodrug” as used herein means the pharmacologically acceptable derivatives of compounds of Formula (I) such as esters whose in vivo biotransformation product is the active drug. Prodrugs are characterized by increased bio-availability and are readily metabolized into the active compounds in vivo. Suitable prodrugs for the purpose of the invention include phosphoramidates, HepDirect, (S)-acyl-2-thioethyl (SATE), carboxylic esters, in particular alkyl esters, aryl esters, acyloxyalkyl esters, and dioxolene carboxylic esters; ascorbic acid esters.

The term “substituent” or “substituted” means that a hydrogen radical on a compound or group is replaced by any desired group which is substantially stable under the reaction conditions in an unprotected form or when protected by a protecting group. Examples of preferred substituents include, without being limited to, halogen (chloro, iodo, bromo, or fluoro); alkyl; alkenyl; alkynyl, as described above; hydroxy; alkoxy; nitro; thiol; thioether; imine; cyano; amido; phosphonato; phosphine; carboxyl; thiocarbonyl; sulfonyl; sulfonamide; ketone; aldehyde; ester; oxygen (—O); haloalkyl (e.g., trifluoromethyl); cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl), monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO₂CH₃; CONH₂; OCH₂CONH₂; NH₂; SO₂NH₂; OCHF₂; CF₃; OCF₃; and such moieties may also be optionally substituted by a fused-ring structure or bridge, for example —OCH₂O—. These substituents may optionally be further substituted with a substituent selected from such groups. In certain embodiments, the term “substituent” or the adjective “substituted” refers to a substituent selected from the group consisting of an alkyl, an alkenyl, an alkynyl, an cycloalkyl, an cycloalkenyl, a heterocycloalkyl, an aryl, a heteroaryl, an arylalkyl, a heteroarylalkyl, a haloalkyl, —C(O)NR₁₇R₁₈, —NR₁₉C(O)R₂₀, a halo, —OR₁₉, cyano, nitro, a haloalkoxy, —C(O)R₁₉, —NR₁₇R₁₈, —SR₁₉, —C(O)OR₁₉, —OC(O)R₁₉, —NR₁₉C(O)NR₁₇R₁₈, —OC(O)NR₁₇R₁₈, —NR₁₉C(O)OR₂₀, —S(O)rR₁₉, —NR₁₉S(O)Rr₂₀, —OS(O)Rr₂₀, S(O)rNR₁₇R₁₈, —O, —S, and —N—R₁₉, wherein r is 1 or 2; R₁₇ and R₁₈, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl; or R₁₇ and R₁₈ taken together with the nitrogen to which they are attached is optionally substituted heterocycloalkyl or optionally substituted heteroaryl; and R₁₉ and R₂₀ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl. In certain embodiments, the term “substituent” or the adjective “substituted” refers to a solubilizing group.

The bonds of an asymmetric carbon can be represented here using a solid triangle ( [symbol] ), a dashed triangle ( [symbol] ) or a zigzag line ( [symbol] ).

The term “active ingredient” or “active agent” refers to a molecule or a substance whose administration to a subject slows down or stops the progression, aggravation, or deterioration of one or more symptoms of a disease, or condition; alleviates the symptoms of a disease or condition; cures a disease or condition. According to one embodiment, the therapeutic ingredient is a small molecule, either natural or synthetic. According to another embodiment, the therapeutic ingredient is a biological molecule such as for example an oligonucleotide, a siRNA, a miRNA, a DNA fragment, an aptamer, an antibody and the like.

By “pharmaceutically acceptable” it is meant that the ingredients of a pharmaceutical composition are compatible with each other and not deleterious to the patient.

The terms “pharmaceutically acceptable excipient”, “pharmaceutically acceptable carrier” or “pharmaceutical vehicle” refer to an inert medium or carrier used as a solvent or diluent in which the pharmaceutically active ingredient is formulated and/or administered, and which does not produce an adverse, allergic or other reaction when administered to an animal, preferably a human being. This includes all solvents, dispersion media, coatings, antibacterial and antifungal agents, isotonic agents, absorption retardants and other similar ingredients. For human administration, preparations must meet standards of sterility, general safety and purity as required by regulatory agencies such as the FDA or EMA. For the purposes of the invention, “pharmaceutically acceptable excipient” includes all pharmaceutically acceptable excipients as well as all pharmaceutically acceptable carriers, diluents, and/or adjuvants.

The term “pharmaceutically acceptable salts” include the acid addition and base salts. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminum, arginine, benzathine, calcium, choline, diethylamine, 2-(diethylamino)ethanol, diolamine, ethanolamine, glycine, 4-(2-hydroxyethyl)-morpholine, lysine, magnesium, meglumine, morpholine, olamine, potassium, sodium, tromethamine and zinc salts.

Hemisalts of acids and bases may also be formed, for example, hemisulphate and hemicalcium salts.

Pharmaceutically acceptable salts of compounds of Formula (I) may be prepared by one or more of these methods:
(i) by reacting the compound of Formula (I) with the desired acid;
(ii) by reacting the compound of Formula (I) with the desired base;
(iii) by removing an acid- or base-labile protecting group from a suitable precursor of the compound of Formula (I) or by ring-opening a suitable cyclic precursor, e.g., a lactone or lactam, using the desired acid; and/or
(iv) by converting one salt of the compound of Formula (I) to another by reaction with an appropriate acid or by means of a suitable ion exchange column.

All these reactions are typically carried out in solution. The salt may precipitate from solution and be collected by filtration or may be recovered by evaporation of the solvent. The degree of ionization in the salt may vary from completely ionized to almost non-ionized.

Although generally, with respect to the salts of the compounds of the invention, pharmaceutically acceptable salts are preferred, it should be noted that the invention in its broadest sense also includes non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of Formula (I).

The term “solvate” is used herein to describe a molecular complex comprising a compound of the invention and containing stoichiometric or sub-stoichiometric amounts of one or more pharmaceutically acceptable solvent molecule, such as ethanol. The term ‘hydrate’ refers to a solvate when said solvent is water.

The term “administration”, or a variant thereof (e.g., “administering”), means providing the active agent or active ingredient, alone or as part of a pharmaceutically acceptable composition, to the patient in whom/which the condition, symptom, or disease is to be treated or prevented.

The term “subject” refers to a mammal, preferably a human. According to the present invention, a subject is a mammal, preferably a human, suffering from SAPHO syndrome. In one embodiment, the subject is a “patient”, i.e., a mammal, preferably a human, who/which is awaiting the receipt of, or is receiving medical care or was/is/will be the object of a medical procedure or is monitored for the development of SAPHO syndrome.

The term “human” refers to a subject of both genders and at any stage of development (i.e., neonate, infant, juvenile, adolescent, adult).

The term “therapeutically effective amount” (or more simply an “effective amount”) as used herein refers to the amount of active agent or active ingredient that is aimed at, without causing significant negative or adverse side effects to the subject in need of treatment, preventing, reducing, alleviating or slowing down (lessening) one or more of the symptoms of SAPHO syndrome.

The terms “treat”, “treating” or “treatment”, as used herein, refer to a therapeutic treatment, to a prophylactic (or preventative) treatment, or to both a therapeutic treatment and a prophylactic (or preventative) treatment, wherein the object is to prevent, reduce, alleviate, and/or slow down (lessen) one or more of the symptoms of SAPHO syndrome, in a subject in need thereof. Symptoms of SAPHO syndrome, include, without being limited:
(1) osteoarticular symptoms (i.e. manifestations affecting bones and/or joints), including synovitis, primitive inflammatory osteitis, osteitis, hyperostosis, axial spondyloarthritis, arthritis, enthesitis, and diffuse idiopathic skeletal hyperostosis (DISH)-like non-marginal enthesophytes;
(2) cutaneous symptoms, including acneiform and neutrophilic dermatoses, especially palmoplantar pustulosis, acne, other features of follicular occlusion syndromes such as hidradenitis suppurativa, and pustular psoriasis;
(3) additional symptoms including systemic features such as fatigue and fever, inflammatory bowel disease (IBD), Crohn disease, venous thrombosis, hypertrophic pachymeningitis, uveitis, sciatica, AA amyloidosis with related renal involvement and pleural abnormalities including both parenchymal and pleural changes.
   In one embodiment, “treating” or “treatment” refers to a therapeutic treatment. In another embodiment, “treating” or “treatment” refers to a prophylactic or preventive treatment. In yet another embodiment, “treating” or “treatment” refers to both a prophylactic (or preventative) treatment and a therapeutic treatment. In one embodiment, “treating” or “treatment” refers to the therapeutic treatment of one or more symptoms of SAPHO syndrome and at the same time the prevention of one or more other symptoms of SAPHO syndrome.

### DETAILED DESCRIPTION

This invention thus relates to the use of nicotinamide mononucleotide derivatives for the treatment of SAPHO syndrome. In particular, the present invention relates to nicotinamide mononucleotide derivatives for use in the treatment of SAPHO syndrome, in a subject in need thereof.

### Nicotinamide Mononucleotide Derivatives

In one embodiment, the nicotinamide mononucleotide derivative of the present invention is a compound of Formula (I)

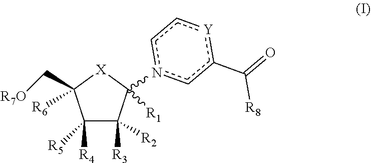

or a pharmaceutically acceptable salt or solvate thereof; wherein:
X is selected from O, CH₂, S, Se, CHF, CF₂ and C═CH₂;
R₁ is selected from H, azido, cyano, (C₁-C₈)alkyl, (C₁-C₈)thio-alkyl, (C₁-C₈)heteroalkyl and OR; wherein R is selected from H and (C₁-C₈)alkyl;
R₂, R₃, R₄ et R₅ are independently selected from H, halogen, azido, cyano, hydroxyl, (C₁-C₁₂)alkyl, (C₁-C₁₂)thio-alkyl, (C₁-C₁₂)heteroalkyl, (C₁-C₁₂)haloalkyl and OR; wherein R is selected from H, (C₁-C₁₂)alkyl, —C(O)(C₁-C₁₂)alkyl, —C(O)NH(C₁-C₁₂)alkyl, —C(O)O(C₁-C₁₂)alkyl, —C(O)aryl, —C(O)(C₁-C₁₂)alkyl-(C₅-C₁₂)aryl, —C(O)NH(C₁-C₁₂)alkyl-(C₅-C₁₂)aryl, —C(O)O(C₁-C₁₂)alkyl-(C₅-C₁₂)aryl and —C(O)CHR_{AA}NH₂; wherein R_{AA} is a side chain selected from a proteinogenic amino acid;
R₆ is selected from H, azido, cyano, (C₁-C₈)alkyl, (C₁-C₈)thio-alkyl, (C₁-C₈)heteroalkyl and OR; wherein R is selected from H and (C₁-C₈)alkyl;
R₇ is selected from H, P(O)R₉R₁₀, P(S)R₉R₁₀ and

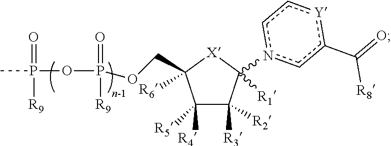

wherein:
   R₉ and R₁₀ are independently selected from OH, OR₁₁, NR₁₃R₁₄, (C₁-C₈)alkyl, (C₂-C₈)alkenyl, (C₂-C₈)alkynyl, (C₃-C₁₀)cycloalkyl, (C₅-C₁₂)aryl, (C₅-C₁₂)aryl-(C₁-C₈)alkyl, (C₁-C₈)alkyl-(C₅-C₁₂)aryl, (C₁-C₈)heteroalkyl, (C₃-C₈)heterocycloalkyl, (C₅-C₁₂)heteroaryl and NHCR_{α}R_{α′}C(O)OR₁₂; wherein:
      R₁₁ is selected from (C₁-C₁₀)alkyl, (C₃-C₁₀)cycloalkyl, (C₅-C₁₂)aryl, (C₁-C₁₀)alkyl-(C₅-C₁₂)aryl, substituted (C₅-C₁₂)aryl, (C₁-C₁₀)heteroalkyl, (C₁-C₁₀)haloalkyl, —(CH₂)ₘC(O)(C₁-C₁₅)alkyl, —(CH₂)ₘOC(O)(C₁-C₁₅)alkyl, —(CH₂)ₘOC(O)O(C₁-C₁₅)alkyl, —(CH₂)ₘSC(O)(C₁-C₁₅)alkyl, —(CH₂)ₘC(O)O(C₁-C₁₅)alkyl, —(CH₂)ₘC(O)O(C₁-C₁₅)alkyl-(C₅-C₁₂)aryl; wherein m is an integer selected from 1 to 8; and —P(O)(OH)OP(O)(OH)₂; and an internal or external counterion;
      R₁₂ is selected from hydrogen, (C₁-C₁₀)alkyl, (C₂-C₈)alkenyl, (C₂-C₈)alkynyl, (C₁-C₁₀)haloalkyl, (C₃-C₁₀)cycloalkyl, (C₃-C₁₀)heterocycloalkyl, (C₅-C₁₂)aryl, (C₁-C₄)alkyl-(C₅-C₁₂)aryl and (C₅-C₁₂)heteroaryl; wherein said aryl or heteroaryl groups are optionally substituted by one or two groups selected from halogen, trifluoromethyl, (C₁-C₆)alkyl, (C₁-C₆)alkoxy and cyano;
      R₁₃ and R₁₄ are independently selected from H, (C₁-C₈)alkyl and (C₁-C₈)alkyl-(C₅-C₁₂)aryl; and
      R_{α} and R_{α′} are independently selected from an hydrogen, (C₁-C₁₀)alkyl, (C₂-C₁₀)alkenyl, (C₂-C₁₀)alkynyl, (C₃-C₁₀)cycloalkyl, (C₁-C₁₀)thio-alkyl, (C₁-C₁₀)hydroxyalkyl, (C₁-C₁₀)alkyl-(C₅-C₁₂)aryl, (C₅-C₁₂)aryl, —(CH₂)₃NHC(═NH)NH₂, (1H-indol-3-yl)methyl, (1H-imidazol-4-yl)methyl and a side chain selected from a proteinogenic or non-proteinogenic amino acid; wherein said aryl groups are optionally substituted with a group selected from hydroxyl, (C₁-C₁₀)alkyl, (C₁-C₆)alkoxy, halogen, nitro and cyano; or
   R₉ and R₁₀ together with the phosphorus atom to which they are attached form a 6-membered ring wherein —R₉-R₁₀— represents —O—CH₂—CH₂—CHR—O—; wherein R is selected from hydrogen, (C₅-C₆)aryl and (C₅-C₆)heteroaryl; wherein said aryl or heteroaryl groups are optionally substituted by one or two groups selected from halogen, trifluoromethyl, (C₁-C₆)alkyl, (C₁-C₆)alkoxy and cyano;
   X′ is selected from O, CH₂, S, Se, CHF, CF₂ and C═CH₂;
   R_{1′} is selected from H, azido, cyano, (C₁-C₈)alkyl, (C₁-C₈)thio-alkyl, (C₁-C₈)heteroalkyl and OR; wherein R is selected from H and (C₁-C₈)alkyl;
   R_{2′}, R_{3′}, R_{4′} et R_{5′} are independently selected from H, halogen, azido, cyano, hydroxyl, (C₁-C₁₂)alkyl, (C₁-C₁₂)thio-alkyl, (C₁-C₁₂)heteroalkyl, (C₁-C₁₂)haloalkyl and OR; wherein R is selected from H, (C₁-C₁₂)alkyl, —C(O)(C₁-C₁₂)alkyl, —C(O)NH(C₁-C₁₂)alkyl, —C(O)O(C₁-C₁₂)alkyl, —C(O)aryl, —C(O)(C₁-C₁₂)alkyl-(C₅-C₁₂)aryl, —C(O)NH(C₁-C₁₂)alkyl-(C₅-C₁₂)aryl, —C(O)O(C₁-C₁₂)alkyl-(C₅-C₁₂)aryl and —C(O)CHR_{AA}NH₂; wherein R_{AA} is a side chain selected from a proteinogenic amino acid;
   R_{6′} is selected from H, azido, cyano, (C₁-C₈)alkyl, (C₁-C₈)thio-alkyl, (C₁-C₈)heteroalkyl and OR; wherein R is selected from H and (C₁-C₈)alkyl;
   R_{8′} is selected from H, OR, NR_{15′}R_{16′}, NH—NHR_{15′}, SH, CN, N₃ and halogen; wherein R is selected from H and (C₁-C₈)alkyl, and R_{15′} and R_{16′} are independently selected from H, (C₁-C₈)alkyl and (C₁-C₈)alkyl-(C₅-C₁₂)aryl and —CHR_{AA′}CO₂H wherein R_{AA′} is a side chain selected from a proteinogenic or non-proteinogenic amino acid;
   Y′ is selected from CH, CH₂, CHCH₃, C(CH₃)₂ and CCH₃;
   n is an integer selected from 1 to 3;
   [symbol] represents the point of attachment;
   [symbol] represents a single or double bond depending on Y′; and
   [symbol] represents the alpha or beta anomer depending on the position of R_{1′};
R₈ is selected from H, OR, NR₁₅R₁₆, NH—NHR₁₅, SH, CN, N₃ and halogen; wherein R is selected from H and (C₁-C₈)alkyl, and R₁₅ and R₁₆ are independently selected from H, (C₁-C₈)alkyl, (C₁-C₈)alkyl-aryl and —CHR_{AA}CO₂H wherein R_{AA} is a side chain selected from a proteinogenic or non-proteinogenic amino acid;
Y is selected from CH, CH₂, CHCH₃, C(CH₃)₂ and CCH₃;
[symbol] represents a single or double bond depending on Y; and
[symbol] represents the alpha or beta anomer depending on the position of R₁.

The nicotinamide mononucleotide derivatives of the invention may comprise one or more charged atoms. Particularly, when present, the phosphate groups may bear one or more charge, preferably one or more negative charge. Moreover, the nitrogen atom of the pyridine part of the nicotinamide group may bear one positive charge when it is quaternized. The presence of one or more charged atom in the nicotinamide mononucleotide derivatives of the invention depends on the conditions, especially pH conditions, that one skilled in the art will recognize.

According to one embodiment, X is selected from O, CH₂ and S. In one embodiment, X is oxygen.

According to one embodiment, R₁ is selected from hydrogen and OH. In one embodiment, R₁ is hydrogen. In one embodiment, R₁ is OH.

According to one embodiment, R₂, R₃, R₄ and R₅ are independently selected from hydrogen, halogen, hydroxyl, (C₁-C₁₂)alkyl and OR; wherein R is as described herein above. In a preferred embodiment, R₂, R₃, R₄ and R₅ are independently selected from hydrogen, hydroxyl and OR; wherein R is as described herein above. In a more preferred embodiment R₂, R₃, R₄ and R₅ are independently selected from hydrogen and OH.

According to one embodiment, R₂ and R₃ are identical. In one embodiment, R₂ and R₃ are identical and represent OH. In one embodiment, R₂ and R₃ are identical and represent hydrogen.

According to one embodiment, R₂ and R₃ are different. In a preferred embodiment, R₂ is hydrogen and R₃ is OH. In a more preferred embodiment, R₂ is OH and R₃ is hydrogen.

According to one embodiment, R₄ and R₅ are identical. In one embodiment, R₄ and R₅ are identical and represent OH. In one embodiment, R₄ and R₅ are identical and represent hydrogen.

According to one embodiment, R₄ and R₅ are different. In a preferred embodiment, R₄ is OH and R₅ is hydrogen. In a more preferred embodiment, R₄ is hydrogen and R₅ is OH.

According to one embodiment, R₃ and R₄ are different. In one embodiment, R₃ is OH and R₄ is hydrogen. In one embodiment, R₃ is hydrogen and R₄ is OH.

According to one embodiment, R₃ and R₄ are identical. In a preferred embodiment, R₃ and R₄ are identical and represent OH. In a more preferred embodiment, R₃ and R₄ are identical and represent hydrogen.

According to one embodiment, R₂ and R₅ are different. In one embodiment, R₂ is hydrogen and R₅ is OH. In one embodiment, R₂ is OH and R₅ is hydrogen.

According to one embodiment, R₂ and R₅ are identical. In a preferred embodiment, R₂ and R₅ are identical and represent hydrogen. In a more preferred embodiment, R₂ and R₅ are identical and represent OH.

According to one embodiment, R₆ is selected from hydrogen and OH. In one embodiment, R₆ is OH. In a preferred embodiment, R₆ is hydrogen.

According to one embodiment, R₇ is selected from hydrogen, P(O)R₉R₁₀ and

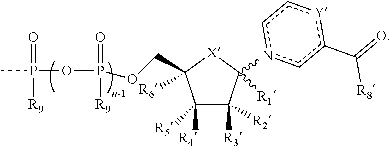

According to one embodiment, R₇ is selected from hydrogen and P(O)R₉R₁₀; wherein R₉ and R₁₀ are as described herein above.

According to one embodiment, R₇ is hydrogen.

According to one embodiment, R₇ is P(O)R₉R₁₀; wherein R₉ and R₁₀ are as described herein above. In a preferred embodiment, R₇ is P(O)(OH)₂.

According to another embodiment, R₇ is

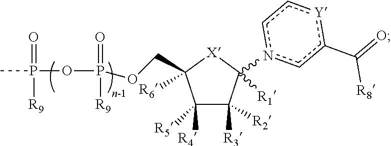

wherein R_{1′}, R_{2′}, R_{3′}, R_{4′}, R_{5′}, R_{6′}, R_{8′}, R₉, X′, Y′, n, [symbol] , [symbol] and [symbol] are as described herein above for compounds of Formula (I).

According to a preferred embodiment, R₇ is

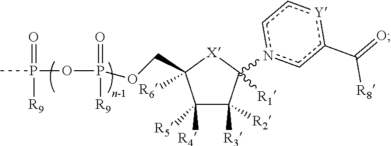

wherein:
X′ is selected from O, CH₂ and S, preferably X′ is O;
R_{1′} is selected from hydrogen and OH, preferably R_{1′} is hydrogen;
R_{2′}, R_{3′}, R_{4′} and R_{5′} are independently selected from hydrogen, halogen, hydroxyl, (C₁-C₁₂)alkyl and OR; wherein R is as described herein above, preferably R₂, R_{3′}, R_{4′} and R_{5′} are independently selected from hydrogen, hydroxyl and OR; wherein R is as described herein above, more preferably R_{2′}, R_{3′}, R_{4′} and R_{5′} are independently selected from hydrogen and OH;
R_{6′} is selected from hydrogen and OH, preferably R_{6′} is hydrogen;
R_{8′} is selected from H, OR and NR₁₅R_{16′}; wherein R_{15′} and R_{16′} are as described herein above, preferably R_{8′} is NHR_{15′}; wherein R_{15′} is as described herein above, more preferably R_{8′} is NH₂;
Y′ is selected from CH and CH₂;
n is an integer selected from 1 to 3;
[symbol] represents the point of attachment;
[symbol] represents a single or double bond depending on Y′; and
[symbol] represents the alpha or beta anomer depending on the position of R_{1′}.

According to one embodiment, in Formula (I),
R₇ is

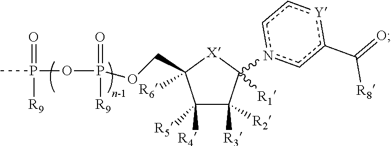

X and X′ are independently selected from O, CH₂ and S, preferably X and X′ are O;
R₁ and R_{1′} are independently selected from hydrogen and OH, preferably R₁ and R_{1′} are hydrogen;
R₂, R₃, R₄, R₅, R_{2′}, R_{3′}, R_{4′} and R_{5′} are independently selected from hydrogen, halogen, hydroxyl, (C₁-C₁₂)alkyl and OR; wherein R is as described herein above, preferably R₂, R₃, R₄, R₅, R_{2′}, R_{3′}, R_{4′} and R_{5′} are independently selected from hydrogen, hydroxyl and OR; wherein R is as described herein above, more preferably R₂, R₃, R₄, R₅, R_{2′}, R_{3′}, R_{4′} and R_{5′} are independently selected from hydrogen and OH;
R₆ and R_{6′} are independently selected from hydrogen and OH, preferably R₆ and R_{6′} are hydrogen;
R₈ and R_{8′} are independently selected from H, OR and NR₁₅R_{16′}; wherein R_{15′} and R_{16′} are as described herein above, preferably R₈ and R_{8′} are NHR_{15′}; wherein R_{15′} is as described herein above, more preferably R₈ and R_{8′} are NH₂;
Y and Y′ are independently selected from CH and CH₂;
n is an integer selected from 1 to 3;
[symbol] represents the point of attachment;
[symbol] represents a single or double bond depending on Y and Y′; and
[symbol] represents the alpha or beta anomers depending on the position of R₁ and R_{1′}.

According to one embodiment, n is 1. According to one embodiment, n is 2. According to one embodiment, n is 3.

According to one embodiment, R₈ is selected from H, OR and NR₁₅R₁₆; wherein R₁₅ and R₁₆ are as described herein above. In a preferred embodiment, R₈ is NHR₁₅; wherein R₁₅ is as described herein above. In one embodiment. R₈ is NH₂.

According to one embodiment, Y is a CH or CH₂. In one embodiment, Y is a CH. In one embodiment, Y is a CH₂.

According to a preferred embodiment, the nicotinamide mononucleotide derivative used in the present invention is of general Formula (II):

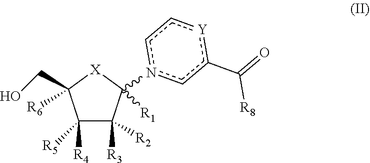

or a pharmaceutically acceptable salt or solvate thereof; wherein R₁, R₂, R₃, R₄, R₅, R₆, R₈, X, Y, [symbol] and [symbol] are as described herein above for compounds of Formula (I).

According to one embodiment, preferred compounds of general Formula (II) are those of Formula (II-1):

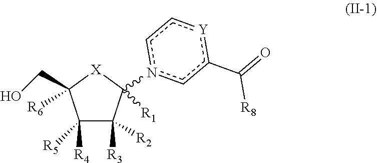

or a pharmaceutically acceptable salt or solvate thereof; wherein R₁, R₂, R₃, R₄, R₅, R₆, R₈, Y, [symbol] and [symbol] are as described herein above for compounds of Formula (I).

According to one embodiment, preferred compounds of general Formula (II) are those of Formula (II-2):

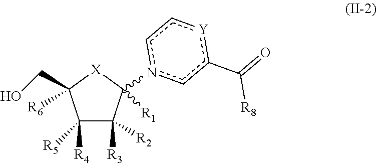

or a pharmaceutically acceptable salt or solvate thereof; wherein R₂, R₃, R₄, R₅, R₆, R₈, Y, [symbol] and [symbol] are as described herein above for compounds of Formula (I).

According to one embodiment, preferred compounds of general Formula (II) are those of Formula (II-3):

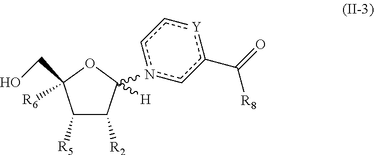

or a pharmaceutically acceptable salt or solvate thereof; wherein R₂, R₅, R₆, R₈, Y, [symbol] and [symbol] are as described herein above for compounds of Formula (I).

According to one embodiment, preferred compounds of general Formula (II) are those of Formula (II-4):

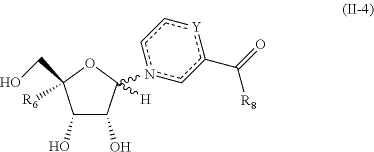

or a pharmaceutically acceptable salt or solvate thereof; wherein R₆, R₈, Y, [symbol] and [symbol] are as described herein above for compounds of Formula (I).

According to one embodiment, preferred compounds of general Formula (II) are those of Formula (II-5):

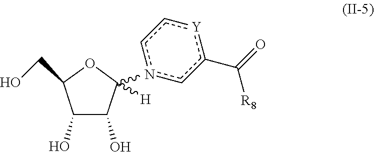

or a pharmaceutically acceptable salt or solvate thereof; wherein R₈, Y, [symbol] and [symbol] are as described herein above for compounds of Formula (I).

According to one embodiment, preferred compounds of general Formula (II) are those of Formula (II-6):

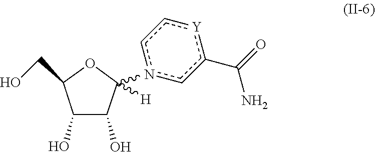

or a pharmaceutically acceptable salt or solvate thereof; wherein Y, [symbol] and [symbol] are as described herein above for compounds of Formula (I).

According to one embodiment, preferred compounds of general Formula (II) are those of Formula (II-7):

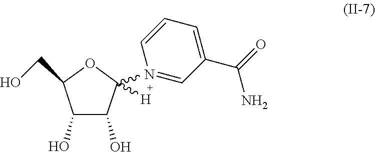

or a pharmaceutically acceptable salt or solvate thereof; wherein [symbol] is as described herein above for compounds of Formula (I).

According to a preferred embodiment, the invention relates to compounds of general Formula (II-8):

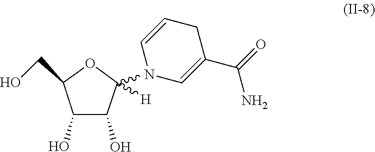

or a pharmaceutically acceptable salt or solvate thereof; wherein [symbol] is as described herein above for compounds of Formula (I).

According to another preferred embodiment, the nicotinamide mononucleotide derivative used in the present invention is of general Formula (III):

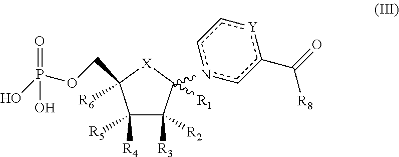

or a pharmaceutically acceptable salt or solvate thereof; wherein R₁, R₂, R₃, R₄, R₅, R₆, R₈, X, Y, [symbol] and [symbol] are as described herein above for compounds of Formula (I).

According to one embodiment, preferred compounds of general Formula (III) are those of Formula (III-1):

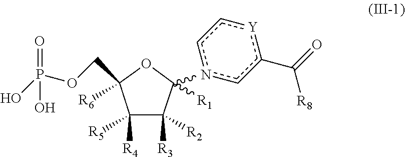

or a pharmaceutically acceptable salt or solvate thereof; wherein R₁, R₂, R₃, R₄, R₅, R₆, R₈, Y, [symbol] and [symbol] are as described herein above for compounds of Formula (I).

According to one embodiment, preferred compounds of general Formula (III) are those of Formula (III-2):

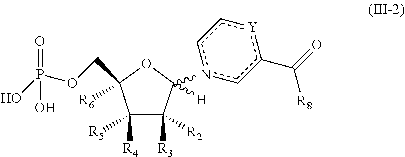

or a pharmaceutically acceptable salt or solvate thereof; wherein R₂, R₃, R₄, R₅, R₆, R₈, Y, [symbol] and [symbol] are as described herein above for compounds of Formula (I).

According to one embodiment, preferred compounds of general Formula (III) are those of Formula (III-3):

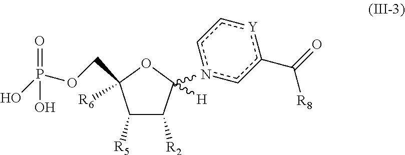

or a pharmaceutically acceptable salt or solvate thereof; wherein R₂, R₅, R₆, R₈, Y, [symbol] and [symbol] are as described herein above for compounds of Formula (I).

According to one embodiment, preferred compounds of general Formula (III) are those of Formula (III-4):

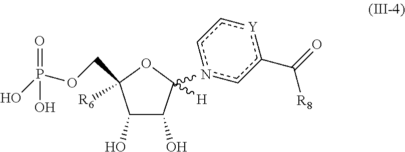

or a pharmaceutically acceptable salt or solvate thereof; wherein R₆, R₈, Y, [symbol] and [symbol] are as described herein above for compounds of Formula (I).

According to one embodiment, preferred compounds of general Formula (III) are those of Formula (III-5):

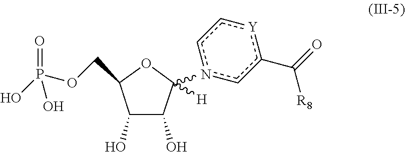

or a pharmaceutically acceptable salt or solvate thereof; wherein R₈, Y, [symbol] and [symbol] are as described herein above for compounds of Formula (I).

According to one embodiment, preferred compounds of general Formula (III) are those of Formula (III-6):

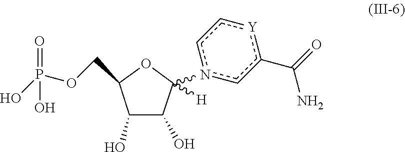

or a pharmaceutically acceptable salt or solvate thereof; wherein Y, [symbol] and [symbol] are as described herein above for compounds of Formula (I).

According to one embodiment, preferred compounds of general Formula (III) are those of Formula (III-7):

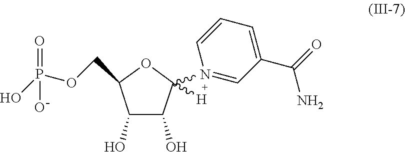

or a pharmaceutically acceptable salt or solvate thereof; wherein [symbol] is as described herein above for compounds of Formula (I).

According to one embodiment, preferred compounds of general Formula (III) are those of Formula (III-8):

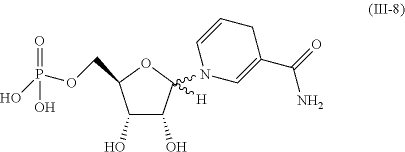

or a pharmaceutically acceptable salt or solvate thereof; wherein [symbol] is as described herein above for compounds of Formula (I).

According to another preferred embodiment, the nicotinamide mononucleotide derivative used in the present invention is of general Formula (IV):

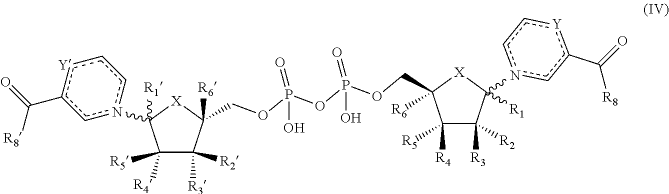

or a pharmaceutically acceptable salt or solvate thereof; wherein R₁, R_{1′}, R₂, R_{2′}, R₃, R_{3′}, R₄, R_{4′}, R₅, R_{5′}, R₆, R_{6′}, R₈, R_{8′}, X, X′, Y, Y′, [symbol] and [symbol] are as described herein above for compounds of Formula (I).

According to one embodiment, preferred compounds of general Formula (IV) are those of Formula (IV-1):

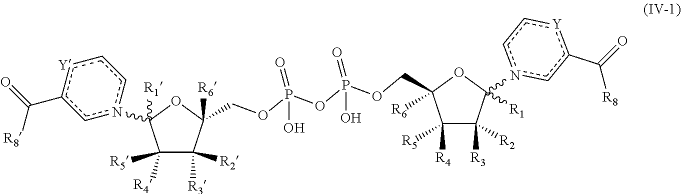

or a pharmaceutically acceptable salt or solvate thereof; wherein R₁, R_{1′}, R₂, R_{2′}, R₃, R_{3′}, R₄, R_{4′}, R₅, R_{5′}, R₆, R_{6′}, R₈, R_{8′}, Y, Y′, [symbol] and [symbol] are as described herein above for compounds of Formula (I).

According to one embodiment, preferred compounds of general Formula (IV) are those of Formula (IV-2):

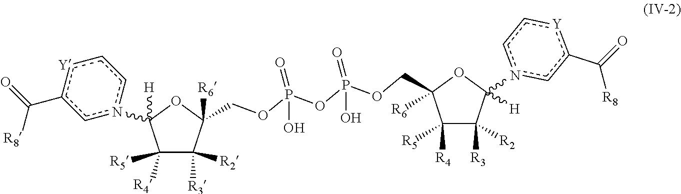

or a pharmaceutically acceptable salt or solvate thereof; wherein R₂, R_{2′}, R₃, R_{3′}, R₄, R_{4′}, R₅, R_{5′}, R₆, R_{6′}, R₈, R_{8′}, Y, Y′, [symbol] and [symbol] are as described herein above for compounds of Formula (I).

According to one embodiment, preferred compounds of general Formula (IV) are those of Formula (IV-3):

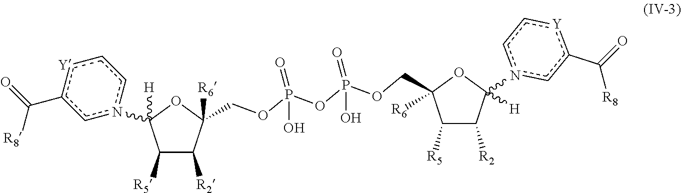

or a pharmaceutically acceptable salt or solvate thereof; wherein R₂, R_{2′}, R₅, R_{5′}, R₆, R_{6′}, R₈, R_{5′}, Y, Y′, [symbol] and [symbol] are as described herein above for compounds of Formula (I).

According to one embodiment, preferred compounds of general Formula (IV) are those of Formula (IV-4):

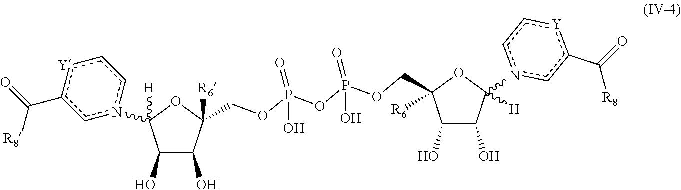

or a pharmaceutically acceptable salt or solvate thereof; wherein R₆, R_{6′}, R₈, R_{8′}, Y, Y′, [symbol] and [symbol] are as described herein above for compounds of Formula (I).

According to one embodiment, preferred compounds of general Formula (IV) are those of Formula (IV-5):

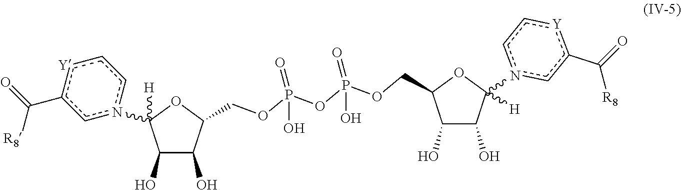

or a pharmaceutically acceptable salt or solvate thereof; wherein R₈, R_{8′}, Y, Y′, [symbol] and [symbol] are as described herein above for compounds of Formula (I).

According to one embodiment, preferred compounds of general Formula (IV) are those of Formula (IV-6):

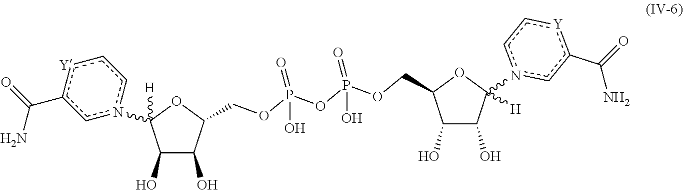

or a pharmaceutically acceptable salt or solvate thereof; wherein Y, Y′, [symbol] and [symbol] are as described herein above for compounds of Formula (I).

According to one embodiment, preferred compounds of general Formula (IV) are those of Formula (IV-7):

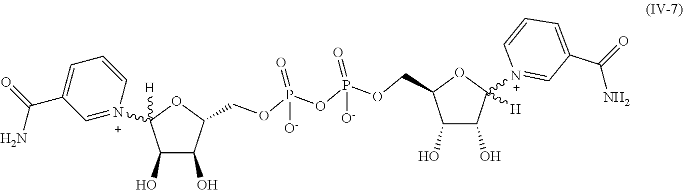

or a pharmaceutically acceptable salt or solvate thereof; wherein [symbol] is as described herein above for compounds of Formula (I).

According to one embodiment, preferred compounds of general Formula (IV) are those of Formula IV-8):

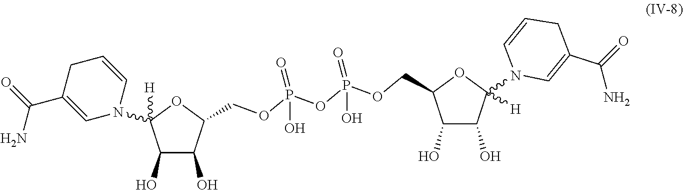

or a pharmaceutically acceptable salt or solvate thereof; wherein [symbol] is as described herein above for compounds of Formula (I).

According to one embodiment, the nicotinamide mononucleotide derivative used in the present invention is selected from compounds 001 to 014 from Table 1 below and pharmaceutically acceptable salts and solvates thereof:

TABLE 1

| Compounds | |
|---|---|
| (anomers) | Structure |
| 001 (beta) NMN | 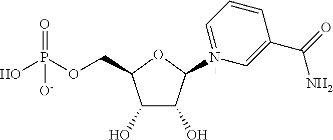 |
| 002 (alpha) | 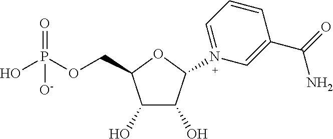 |
| 003 (beta) | 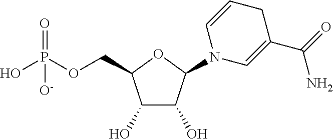 [SMILES: NC(=O)C1=CN([C@@H]2O[C@H](COP(=O)([O-])O)[C@@H](O)[C@H]2O)C=CC1] |
| 004 (alpha) | 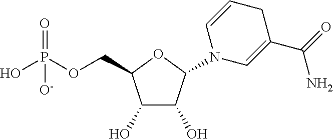 [SMILES: NC(=O)C1=CN([C@H]2O[C@H](COP(=O)([O-])O)[C@@H](O)[C@H]2O)C=CC1] |
| 005 (beta) | 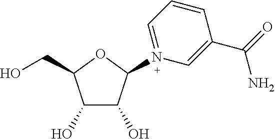 |
| 006 (alpha) | 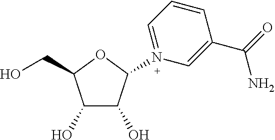 |
| 007 (beta) | 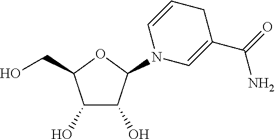 [SMILES: NC(=O)C1=CN([C@@H]2O[C@H](CO)[C@@H](O)[C@H]2O)C=CC1] |
| 008 (alpha) | 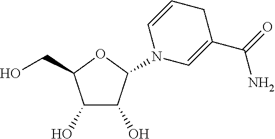 [SMILES: NC(=O)C1=CN([C@H]2O[C@H](CO)[C@@H](O)[C@H]2O)C=CC1] |
| 009 (beta, beta) | 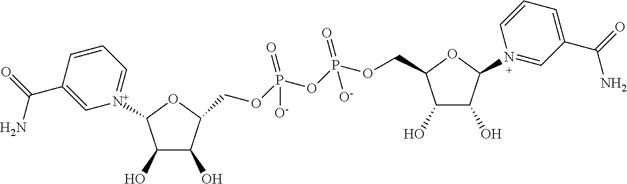 |
| 010 (beta, alpha) | 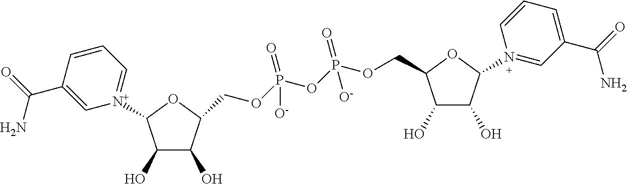 |
| 011 (alpha, alpha) | 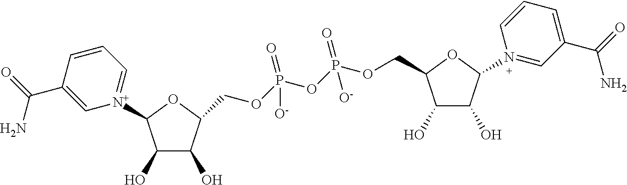 |
| 012 (beta, beta) | 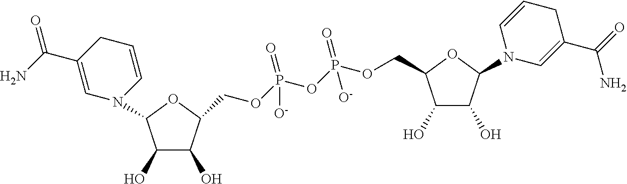 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| 013 (beta, alpha) | 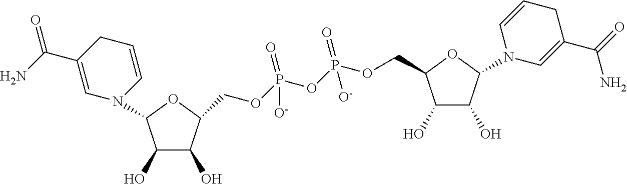 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| 014 (alpha, alpha) | 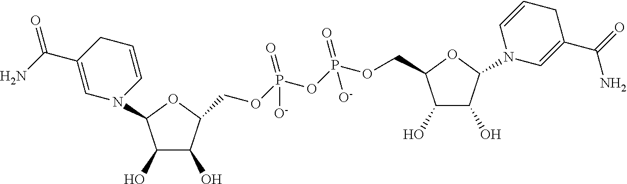 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |

According to one embodiment, preferred nicotinamide mononucleotide derivatives are compounds 001 to 014 or a pharmaceutically acceptable salt or solvate thereof.

According to one embodiment, more preferred nicotinamide mononucleotide derivatives are compounds 001, 002, 009, 010 and 011 or a pharmaceutically acceptable salt or solvate thereof.

According to one embodiment, more preferred nicotinamide mononucleotide derivatives are compounds 001 and 002 or a pharmaceutically acceptable salt or solvate thereof.

According to another embodiment, more preferred nicotinamide mononucleotide derivatives are compounds 009, 010 and 011 or a pharmaceutically acceptable salt or solvate thereof.

According to one embodiment, even more preferred nicotinamide mononucleotide derivatives are compounds 002, 010 and 011 or a pharmaceutically acceptable salt or solvate thereof.

According to one embodiment, the nicotinamide mononucleotide derivatives are compounds 001 to 008 or a pharmaceutically acceptable salt or solvate thereof.

All references to compounds of Formula (I) and subformulae thereof include references to salts, solvates, multi-component complexes, liquid crystals thereof. All references to compounds of Formula (I) and subformulae thereof include references to polymorphs and crystal habits thereof. All references to compounds of Formula (I) and subformulae thereof include references to pharmaceutically acceptable prodrugs thereof.

The nicotinamide mononucleotide derivatives used in the present invention can be under the form of a pharmaceutical composition. In one embodiment, the pharmaceutical composition comprises a nicotinamide mononucleotide derivative as defined hereinabove, and at least one pharmaceutically acceptable carrier.

### Synthesis of the Nicotinamide Mononucleotide Derivatives

According to one embodiment, the nicotinamide mononucleotide derivatives herein defined, especially compounds of Formula (I) and subformulae thereof, can be prepared by any suitable process known in the art.

The invention also provides a method for the preparation of the compound of Formula (I). In particular, the compounds of Formula (I) may be prepared as described below from substrates A-E. It shall be understood by a person skilled in the art that these schemes are in no way limiting and that variations may be made without departing from the spirit and scope of this invention.

According to one embodiment, the method involves in a first step the mono-phosphorylation of a compound of Formula (A), in the presence of phosphoryl chloride and a trialkyl phosphate, to yield the phosphorodichloridate of Formula (B):

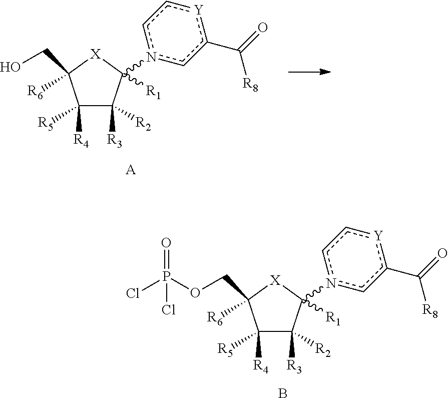

wherein X, R₁, R₂, R₃, R₄, R₅, R₆, R₈, Y, [symbol] and [symbol] are as described herein above.

In a second step, the phosphorodichloridate of Formula (B) is hydrolyzed to yield the phosphate of Formula (C):

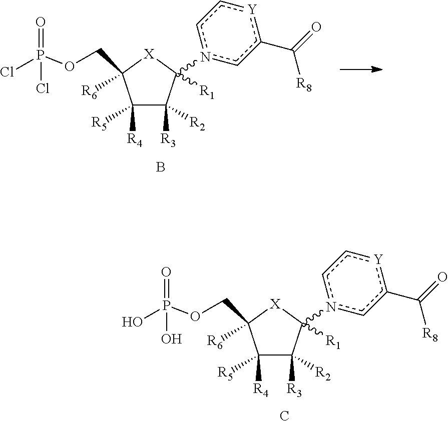

wherein X, R₁, R₂, R₃, R₄, R₅, R₆, R₈, Y, [symbol] and [symbol] are as described herein above.

In an alternative embodiment, when in Formula (I) R₇ is

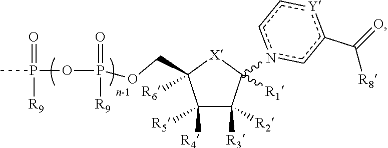

the phosphate compound of Formula (C) obtained in the second step is then reacted with a phosphorodichloridate compound of Formula (B′) obtained as described in the first step:

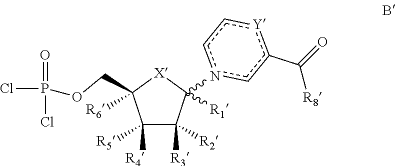

wherein R_{1′}, R_{2′}, R_{3′}, R_{4′}, R_{5′}, R_{6′}, R_{8′}, X′, Y′, [symbol] and [symbol] are as described herein above; to give the compound of Formula (I) as described herein above; followed by hydrolysis to yield to compound of Formula (I).

According to one embodiment, the compound of Formula (A) is synthesized using various methods known to the person skilled in the art.

According to one embodiment, the compound of Formula (A) wherein Y is CH, referred to as compound of Formula (A-a), is synthesized by reacting the pentose of Formula (D) with a nicotinamide derivative of Formula (E), leading to the compound of Formula (A-1), which is then selectively deprotected to give the compound of Formula (A-a):

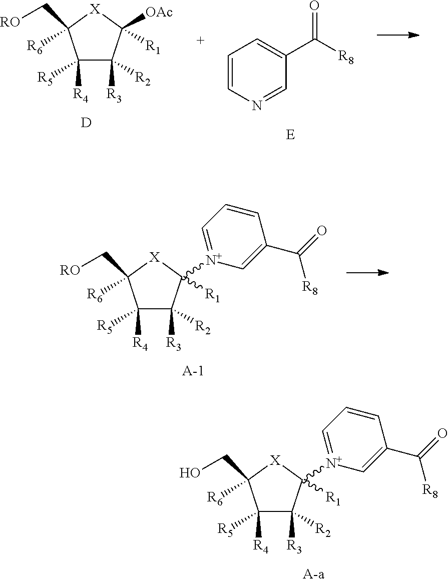

wherein X, R₁, R₂, R₃, R₄, R₅, R₆, R₈, Y, [symbol] and [symbol] are as described herein above and R is a protective group.

According to one embodiment, R is an appropriate protective group known to the skilled person in the art. In one embodiment, the protecting group is selected from triarylmethyls and silyls. Non-limiting examples of triarylmethyl include trityl, monomethoxytrityl, 4,4′-dimethoxytrityl and 4,4′,4″-trimethoxytrityl. Non-limiting examples of silyl groups include trimethylsilyl, tert-butyldimethylsilyl, triisopropylsilyl, tert-butyldiphenylsilyl, tri-iso-propylsilyloxymethyl and [2-(trimethylsilyl)ethoxy]methyl.

According to one embodiment, any hydroxyl group attached to the pentose is protected by an appropriate protective group known to the person skilled in the art.

The choice and exchange of protective groups is the responsibility of the person skilled in the art. Protective groups can also be removed by methods well known to the skilled person, for example, with an acid (e.g. mineral or organic acid), base or fluoride source.

According to a preferred embodiment, the nitrogen nicotinamide of Formula (E) is coupled to the pentose of Formula (D) by a reaction in the presence of a Lewis acid leading to the compound of Formula (A-1). Non-limiting examples of Lewis acids include TMSOTf, BF₃·OEt₂, TiCl₄ and FeCl₃.

According to one embodiment, the method of the present invention further comprises a step of reducing the compound of Formula (A-a) by various methods well known to the skilled person in the art, leading to the compound of Formula (A-b) wherein Y is CH₂ and X, R₁, R₂, R₃, R₄, R₅, R₆, R₈, [symbol] and [symbol] are as defined above.

According to a specific embodiment, the present invention relates to a method for the preparation of the compounds 001, 003, 005, 007 and 009:

In a first step, the nicotinamide of Formula (E-i) is coupled to the ribose tetraacetate of Formula (D-i) by a coupling reaction in the presence of a Lewis acid, resulting in the compound of Formula (A-1-i):

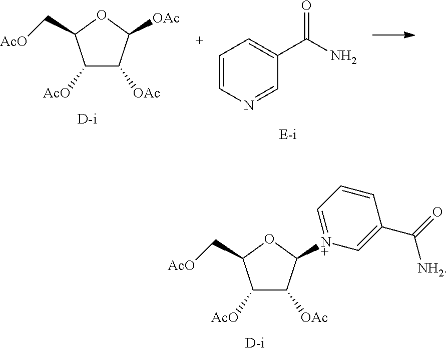

In a second step, an ammoniacal treatment of the compound of Formula (A-1-i) is carried out, leading to the compound 005:

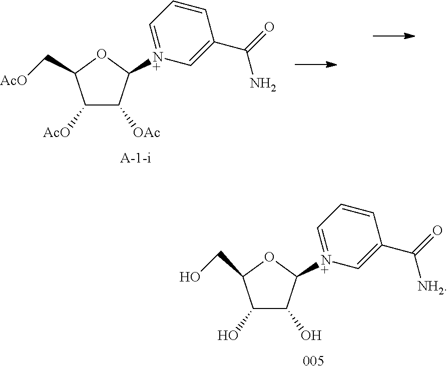

In a third step, the mono-phosphorylation of compound 005, in the presence of phosphoryl chloride and a trialkyl phosphate, leads to the phosphorodichloridate of Formula (B-i):

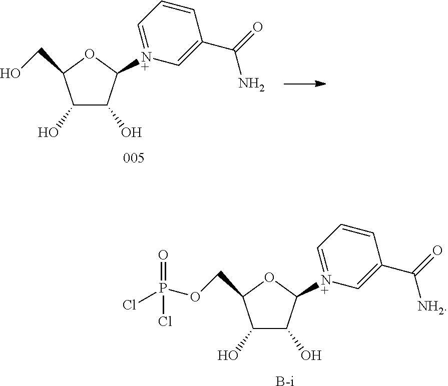

In a fourth step, the phosphorodichloridate of Formula (B-i) is hydrolyzed to yield the compound 001:

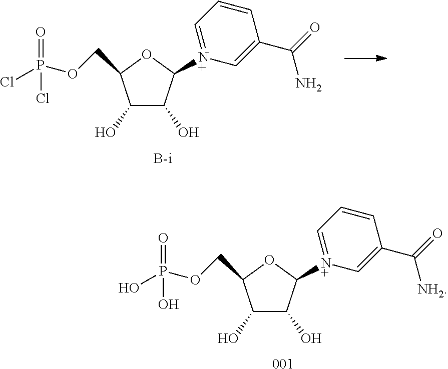

Alternatively, in a fifth step, the phosphate compound 001 obtained in the fourth step is then reacted, with the phosphorodichloridate compound of Formula (B-i) obtained as described in the third step, to give the compound 009.

According to one embodiment, a step of reducing the compound 005 is carried out, leading to the compound 007.

The compound 007 is then monophosphorylated as described in the fourth step and hydrolyzed to the compound 003.

The above method for the preparation of the compounds 001, 003, 005 and 007 can be easily adapted to the synthesis of compounds 002, 004, 006 and 008 by using the suitable starting ribose tetraacetate of Formula (D-ii):

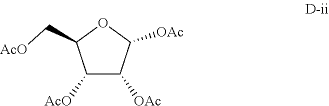 [SMILES: CC(=O)OC[C@H]1O[C@H](OC(C)=O)[C@H](OC(C)=O)[C@@H]1OC(C)=O]

The above method for the preparation of the dimer compound 009 can be easily adapted to the synthesis of dimer compounds 010-014 by using corresponding suitable phosphorodichloridate and phosphate intermediates.

### Treatment of SAPHO Syndrome

As mentioned above, there is an unmet need for the treatment of SAPHO syndrome, especially for the concomitant treatment of at least two symptoms of SAPHO syndrome. This is thus an object of the present invention to provide a treatment of SAPHO syndrome for subjects in need thereof. Especially, the present invention relates to the nicotinamide mononucleotide derivatives defined hereinabove for use in the treatment of SAPHO syndrome in a subject in need thereof.

### SAPHO Syndrome Condition

“SAPHO syndrome” refers to a rare disease which was first described in 1987, with a prevalence estimated to be less than 1 to 10,000. It encompasses several chronic conditions that affect bones, joints and/or skin. The SAPHO syndrome takes its acronymic name based upon the main manifestation usually observed during the disease: synovitis, acne, pustulosis, hyperostosis, and osteitis. Synovitis refers to the inflammation of the envelope that surrounds the joints and manifests as a swollen, painful and stiff joint. Acne is a condition of the sebaceous hair gland which clogs and forms pimples, especially on the face and in the back. Pustulosis is an inflammatory skin disease characterized by pustules appearing on the soles of the feet or palms of hands, which dry out and then cause peeling of the skin. Hyperostosis is the excessive growth of one or more bones (with thickening then possible deformation). Osteitis is an inflammatory disease of the bone which often results in continual pain.

Unless otherwise stated, references made in the present application to SAPHO syndrome encompass the adult form usually named SAPHO syndrome and the child form usually named chronic recurrent multifocal osteomyelitis (CRMO). In one embodiment, “SAPHO syndrome” relates to both adult and child forms. In another embodiment, “SAPHO syndrome” relates only to the adult form of the syndrome, at the exclusion of the child form CRMO.

There is a wide variability in the osteoarticular and cutaneous manifestations of the SAPHO syndrome, and further manifestations may also be observed. Symptoms of the SAPHO syndrome include:
(1) osteoarticular symptoms (i.e. manifestations affecting bones and/or joints), including synovitis, osteitis, primitive inflammatory osteitis, hyperostosis, axial spondyloarthritis, arthritis, enthesitis and diffuse idiopathic skeletal hyperostosis (DISH)-like non-marginal enthesophytes;
(2) cutaneous symptoms, including acneiform and neutrophilic dermatoses, especially palmoplantar pustulosis, acne, other features of follicular occlusion syndromes such as hidradenitis suppurativa, psoriasis vulgaris and pustular psoriasis;
(3) additional symptoms including systemic features such as fatigue and fever, inflammatory bowel disease (IBD), Crohn disease, venous thrombosis, hypertrophic pachymeningitis, uveitis, sciatica, AA amyloidosis with related renal involvement and pleural abnormalities including both parenchymal and pleural changes.

Bone and joint symptoms may affect a variety of regions, especially the anterior chest wall, other parts of the axial skeleton, including the sacroiliac joint and spine; and medium to large lower-extremity joints.

Cutaneous symptoms, may affect hands and/or feet (particularly in case of palmoplantar pustulosis). Acne, and particularly nodulocystic acne, involves the face, chest, and back.

Patients with SAPHO syndrome do not necessarily have all of the above symptoms at the same time, nor successively. Almost all the combinations of symptoms are possible.

### Subjects in Need of Treatment

Preferably, the subject in need of therapeutic treatment in the present invention is a warm-blooded animal, more preferably a human. According to one embodiment, the subject is a male. According to one embodiment, the subject is a female.

In a preferred embodiment the subject is an adult. In one embodiment, the subject is older than 18, 19, 20 or 21 years of age. In one embodiment, the subject is older than 25 or 30 years of age. In one embodiment, the subject is younger than 60, 55 or 50 years of age. In one embodiment, the patient is 20 to 60, 25 to 55 or 30 to 50 year old. In one embodiment, the patient can also be a child, being younger than 18, 17, 16 or 15 years of age.

In one embodiment, the subject suffers from at least one symptom selected from: osteoarticular symptoms including synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes; and cutaneous symptoms including acneiform and neutrophilic dermatoses, especially palmoplantar pustulosis, acne, other features of follicular occlusion syndromes such as hidradenitis suppurativa, psoriasis vulgaris and pustular psoriasis. In one embodiment, the subject suffers from at least one symptom selected from: osteoarticular symptoms including synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes; and cutaneous symptoms including acneiform and neutrophilic dermatoses, especially palmoplantar pustulosis, acne, other features of follicular occlusion syndromes such as hidradenitis suppurativa, and pustular psoriasis.

In one embodiment, the subject suffers from:
(a) one or more symptoms selected from:
   (1) osteoarticular symptoms (including synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes; and
   (2) cutaneous symptoms, including acneiform and neutrophilic dermatoses, especially palmoplantar pustulosis, acne, other features of follicular occlusion syndromes such as hidradenitis suppurativa, psoriasis vulgaris and pustular psoriasis; and
(b) optionally one or more additional symptoms including systemic features such as fatigue and fever, inflammatory bowel disease (IBD), Crohn disease, venous thrombosis, hypertrophic pachymeningitis, uveitis, sciatica, AA amyloidosis with related renal involvement and pleural abnormalities including both parenchymal and pleural changes.

In one embodiment, the subject suffers from:
(a) one or more symptoms selected from:
   (1) osteoarticular symptoms (including synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes; and
   (2) cutaneous symptoms, including acneiform and neutrophilic dermatoses, especially palmoplantar pustulosis, acne, other features of follicular occlusion syndromes such as hidradenitis suppurativa, and pustular psoriasis; and
(b) optionally one or more additional symptoms including systemic features such as fatigue and fever, inflammatory bowel disease (IBD), Crohn disease, venous thrombosis, hypertrophic pachymeningitis, uveitis, sciatica, AA amyloidosis with related renal involvement and pleural abnormalities including both parenchymal and pleural changes.

In one embodiment, the subject suffers from:
one first symptom which is an osteoarticular symptom of SAPHO syndrome selected from synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes;

and
at least one second symptom of SAPHO syndrome, different from the first symptom, selected from:
   osteoarticular symptoms selected from synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes;
   cutaneous symptoms selected from palmoplantar pustulosis, acne, hidradenitis suppurativa, and pustular psoriasis; and
   symptoms selected from fatigue, fever, inflammatory bowel disease (IBD), Crohn disease, venous thrombosis, hypertrophic pachymeningitis, uveitis, sciatica, AA amyloidosis with related renal involvement, pleural abnormalities with parenchymal changes and pleural abnormalities with pleural changes.

In one embodiment, the subject suffers from two or more osteoarticular and/or cutaneous symptoms of SAPHO syndrome. In one embodiment, the subject suffers from three or more osteoarticular and/or cutaneous symptoms of SAPHO syndrome.

In one embodiment, the subject suffers from one or more osteoarticular symptoms of SAPHO syndrome and from one or more cutaneous symptoms of SAPHO syndrome.

In one embodiment, the subject suffers from a bone symptom, particularly a sterile bone inflammation (which is not caused by bacteria) affecting the thorax, spine or pelvis, for example osteitis or hyperostosis; with or without cutaneous symptoms.

In one embodiment, the subject suffers from a joint symptom, particularly a joint damage such as synovitis; associated with a cutaneous symptom, such as severe acne, psoriasis or palmoplantar pustulosis.

In one embodiment, the subject suffering from SAPHO syndrome does not suffer from classic rheumatic diseases, such as rheumatoid arthritis, axial spondyloarthritis, or psoriatic arthritis. In one embodiment, the subject suffering from SAPHO syndrome does not suffer from psoriatic arthritis.

In one embodiment, the subject suffers from SAPHO syndrome, as defined above. According to one embodiment, the subject in need of treatment of SAPHO syndrome is diagnosed by a health professional. The diagnosis of SAPHO syndrome is usually difficult given the wide variety of clinical manifestations that may occur in the patients.

The diagnosis of SAPHO syndrome usually includes a clinical diagnosis that is based upon the presence of a combination of features. The absence of cutaneous manifestations does not exclude the diagnosis of a SAPHO syndrome. Osteoarticular symptoms can be evaluated using various index, such as for example the Bath Ankylosing Spondylitis Disease Activity Index (BASDAI) or the Bath Ankylosing Spondylitis Functional Index (BASFI).

The diagnosis of SAPHO syndrome can also be based on imaging technics, especially for the osteoarticular manifestations, including radiography, tomography, bone scan (scintigraphy), magnetic resonance imaging (MRI), and positron emission tomography (PET).

The diagnosis of SAPHO syndrome is also a diagnosis of exclusion that requires an absence of evidence to support an infectious, malignant, or other etiology; as well as the exclusion of classic rheumatic diseases, such as rheumatoid arthritis, axial spondyloarthritis, or psoriatic arthritis should also be excluded.

In one embodiment, the subject does not suffer from any underlying condition or disease.

In another embodiment, the subject suffers from at least one underlying condition or disease. Examples of underlying conditions or diseases that may coexist in the subject with SAPHO syndrome include, without being limited to, diabetes, hypertension, and obesity.

### Therapeutic Effect

In one embodiment, the use of a nicotinamide mononucleotide derivative as described above prevents, reduces, alleviates, and/or slows down (lessens) one or more of the symptoms of SAPHO syndrome, especially one or more of the following symptoms:
(1) osteoarticular symptoms (including synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes;
(2) cutaneous symptoms, including acneiform and neutrophilic dermatoses, especially palmoplantar pustulosis, acne, other features of follicular occlusion syndromes such as hidradenitis suppurativa, psoriasis vulgaris and pustular psoriasis; and
(3) additional symptoms including systemic features such as fatigue and fever, inflammatory bowel disease (IBD), Crohn disease, venous thrombosis, hypertrophic pachymeningitis, uveitis, sciatica, AA amyloidosis with related renal involvement and pleural abnormalities including both parenchymal and pleural changes.

In one embodiment, the use of a nicotinamide mononucleotide derivative as described above prevents, reduces, alleviates, and/or slows down (lessens) at least one symptom selected from: osteoarticular symptoms including synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes; and cutaneous symptoms including acneiform and neutrophilic dermatoses, especially palmoplantar pustulosis, acne, other features of follicular occlusion syndromes such as hidradenitis suppurativa, psoriasis vulgaris and pustular psoriasis.

In one embodiment, the use of a nicotinamide mononucleotide derivative as described above prevents, reduces, alleviates, and/or slows down (lessens):
one first symptom which is an osteoarticular symptom of SAPHO syndrome selected from synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes;

and
at least one second symptom of SAPHO syndrome, different from the first symptom, selected from:
   osteoarticular symptoms selected from synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes;
   cutaneous symptoms selected from palmoplantar pustulosis, acne, hidradenitis suppurativa, and pustular psoriasis; and
   symptoms selected from fatigue, fever, inflammatory bowel disease (IBD), Crohn disease, venous thrombosis, hypertrophic pachymeningitis, uveitis, sciatica, AA amyloidosis with related renal involvement, pleural abnormalities with parenchymal changes and pleural abnormalities with pleural changes.

In one embodiment, the use of a nicotinamide mononucleotide derivative as described above prevents, reduces, alleviates, and/or slows down (lessens) two or more osteoarticular and/or cutaneous symptoms of SAPHO syndrome. In one embodiment, the use of a nicotinamide mononucleotide derivative as described above prevents, reduces, alleviates, and/or slows down (lessens) three or more osteoarticular and/or cutaneous symptoms of SAPHO syndrome.

In one embodiment, the use of a nicotinamide mononucleotide derivative as described above prevents, reduces, alleviates, and/or slows down (lessens) one or more osteoarticular symptoms of SAPHO syndrome and one or more cutaneous symptoms of SAPHO syndrome.

In one embodiment, the use of a nicotinamide mononucleotide derivative as described above prevents the onset of new symptoms of SAPHO syndrome.

In one embodiment, the use of a nicotinamide mononucleotide derivative as described above enables to therapeutically treat one or more symptoms of SAPHO syndrome and to prevent the onset of new symptoms of SAPHO syndrome.

In one embodiment, the use of a nicotinamide mononucleotide derivative as described above enables to treat one or more symptoms of SAPHO syndrome and to prevent the worsening of one or more symptom of SAPHO syndrome.

In one embodiment, the use of a nicotinamide mononucleotide derivative as described above brings an improvement in the clinical status of the subject suffering from SAPHO syndrome.

In one embodiment, the use of a nicotinamide mononucleotide derivative as described above prevents clinical status degradation of the subject suffering from SAPHO syndrome.

In one embodiment, the use of a nicotinamide mononucleotide derivative as described above prevents the clinical progression of SAPHO syndrome.

### Method of Administration

The compounds of the invention, as described hereinabove, may be administered by oral, parenteral (e.g., intramuscular, intraperitoneal, intravenous, ICV, intracisternal injection or infusion, subcutaneous injection, or implant), inhalation spray (including nebulization), nasal, rectal, sublingual, or topical routes of administration and may be formulated, alone or together, in suitable dosage unit formulations containing conventional non-toxic pharmaceutically acceptable carriers, adjuvants and vehicles appropriate for each route of administration. The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active ingredient is included in an amount sufficient to produce the desired effect.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated, or they may be coated by known techniques to delay disintegration and absorption in the gastrointestinal tract and thereby provide a sustained action over a longer period. For example, a time delay material, such as glyceryl monostearate or glyceryl distearate may be employed. They may also be coated by the techniques described in the U.S. Pat. Nos. 4,256,108; 4,166,452; and U.S. Pat. No. 4,265,874 to form osmotic therapeutic tablets for control release. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol, such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin. Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavoring agents may be added to provide a palatable oral preparation. These compositions may be preserved by the addition of an anti-oxidant, such as ascorbic acid. Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids, such as oleic acid find use in the preparation of injectables. The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols. For topical use, creams, ointments, jellies, solutions or suspensions, etc., containing the compounds of the present invention are employed. (For purposes of this application, topical application shall include mouthwashes and gargles.)

### Dosing Regimen

In the treatment of SAPHO syndrome, an appropriate dosage level will generally be about 0.01 to 500 mg per kg subject body weight per day which can be administered in single or multiple doses. Preferably, the dosage level will be about 0.1 to about 350 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the subject to be treated.

According to one embodiment, the subject in need thereof receives a treatment of at least one nicotinamide mononucleotide derivative as described above at a cumulative dose, preferably an annual cumulative dose, of greater than 100 mg/kg, 200 mg/kg, 300 mg/kg, 400 mg/kg, 500 mg/kg, 600 mg/kg, 700 mg/kg, 800 mg/kg, 900 mg/kg, 1000 mg/kg, 2500 mg/kg or 5000 mg/kg. In one embodiment, the subject in need thereof receives a treatment of at least one nicotinamide mononucleotide derivative as described above at a cumulative dose, preferably an annual cumulative dose, of greater than 400 mg/kg, 500 mg/kg, 600 mg/kg, 700 mg/kg, 800 mg/kg, 900 mg/kg, 1000 mg/kg, 2500 mg/kg or 5000 mg/kg.

The compounds may be administered on a regimen of 1 to 4 times per day, preferably once, twice or three times per day. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Monotherapy/Combination Therapy

The nicotinamide mononucleotide derivatives may be used in monotherapy or in combination therapy in a subject in need thereof.

According to a first embodiment, the nicotinamide mononucleotide derivative is administered to the subject without any other active ingredient.

According to a second embodiment, the nicotinamide mononucleotide derivative is administered to the subject in combination with at least one additional active ingredient. Additional active ingredients of particular interest are those suitable to treat one or more symptoms of SAPHO syndrome. Examples of additional active ingredients include nonsteroidal anti-inflammatory drugs (NSAIDs), antibiotics, methotrexate, TNF inhibitors, IL-6 inhibitors, retinoids, bisphosphonates, sulfonamides, colchicine, corticosteroids, morphine-type analgesics, caltonin, and food supplements and plant extracts.

Examples of nonsteroidal anti-inflammatory drugs (NSAIDs) include naproxen, ibuprofen, indomethacin, diclofenac, celecoxib, etoricoxib, mefenamic acid, and high dose aspirin.

Examples of antibiotics include tetracyclines (such as doxycycline or minocycline), trimethoprim, sulfamethoxazole, azithromycin, and clindamycin.

Examples of TNF inhibitors include etanercept, adalimumab, infliximab, certolizumab pegol, and golimumab.

Examples of IL-6 inhibitors include tocilizumab, sarilumab, siltuximab, olokizumab, elsilimomab, clazakizumab, sirukumab, and levilimab.

Examples of retinoids include acitretin, isotretinoin, retinol, retinoic acid, adapalene, alitretinoin, bexarotene, and adapalene.

Examples of bisphosphonates include pamidronate, risedronate, alendronate, ibandronate, zoledronic acid, and etidronate.

Examples of sulfonamides include sulfasalazine, sulfomethoxasol, and sulfisoxasol.

Examples of corticosteroids include prednisone, hydrocortisone, prednisolone, dexamethasone, methylprednisolone, triamcinolone, and betamethasone.

Examples of morphine-type analgesics include oxycodone, hydrocodone, codeine, fentanyl, hydromorphone, and oxymorphone.

Examples of food supplements and plant extracts include S-adenosylmethionine, Boswellic acids, capsaicin or Capsicum frutescens, curcumin, turmeric, avocado soybean unsaponifiables, Uncaria tomentosa, fish oil, omega 3 fatty acids (EPA and DHA), gamma linoleic acid, ginger, Zingiber officinale, cannabidiol, CBD, chondroitin, glucosamine, and harpagophytum.

In one embodiment, the at least one additional active ingredient is a pharmaceutically active agent selected from naproxen, ibuprofen, indomethacin, diclofenac, celecoxib, etoricoxib, mefenamic acid, high dose aspirin, doxycycline, minocycline, trimethoprim, sulfamethoxazole, azithromycin, clindamycin, methotrexate, etanercept, adalimumab, infliximab, certolizumab pegol, golimumab, tocilizumab, sarilumab, siltuximab, olokizumab, elsilimomab, clazakizumab, sirukumab, levilimab, acitretin, isotretinoin, retinol, retinoic acid, adapalene, alitretinoin, bexarotene, adapalene, pamidronate, risedronate, alendronate, ibandronate, zoledronic acid, etidronate, sulfasalazine, sulfomethoxasol, sulfisoxasol, colchicine, prednisone, hydrocortisone, prednisolone, dexamethasone, methylprednisolone, triamcinolone, betamethasone, oxycodone, hydrocodone, codeine, fentanyl, hydromorphone, oxymorphone, and caltonin.

In one embodiment, the at least one additional active ingredient is a food supplement or plant extract selected from S-adenosylmethionine, Boswellic acids, capsaicin or Capsicum frutescens, curcumin, turmeric, avocado soybean unsaponifiables, Uncaria tomentosa, fish oil, omega 3 fatty acids (EPA and DHA), gamma linoleic acid, ginger, Zingiber officinale, cannabidiol, CBD, chondroitin, glucosamine, and harpagophytum.

In one embodiment, the nicotinamide mononucleotide derivative is administered to the subject sequentially, simultaneously and/or separately with the additional active ingredient, further food supplement or/or plant extract.

In one embodiment, the nicotinamide mononucleotide derivative is administered simultaneously, separately or sequentially with at least one further pharmaceutically active agent selected from naproxen, ibuprofen, indomethacin, diclofenac, celecoxib, etoricoxib, mefenamic acid, high dose aspirin, doxycycline, minocycline, trimethoprim, sulfamethoxazole, azithromycin, clindamycin, methotrexate, etanercept, adalimumab, infliximab, certolizumab pegol, golimumab, tocilizumab, sarilumab, siltuximab, olokizumab, elsilimomab, clazakizumab, sirukumab, levilimab, acitretin, isotretinoin, retinol, retinoic acid, adapalene, alitretinoin, bexarotene, adapalene, pamidronate, risedronate, alendronate, ibandronate, zoledronic acid, etidronate, sulfasalazine, sulfomethoxasol, sulfisoxasol, colchicine, prednisone, hydrocortisone, prednisolone, dexamethasone, methylprednisolone, triamcinolone, betamethasone, oxycodone, hydrocodone, codeine, fentanyl, hydromorphone, oxymorphone, and caltonin; and/or with at least one further food supplement or plant extract selected from S-adenosylmethionine, Boswellic acids, capsaicin or Capsicum frutescens, curcumin, turmeric, avocado soybean unsaponifiables, Uncaria tomentosa, fish oil, omega 3 fatty acids (EPA and DHA), gamma linoleic acid, ginger, Zingiber officinale, cannabidiol, CBD, chondroitin, glucosamine, and harpagophytum.

### Kit of Parts

Another object of the invention is a kit-of-parts comprising a first part comprising a nicotinamide mononucleotide derivative as described hereinabove, and a second part comprising another active ingredient, e.g., an active ingredient selected from but not limited to nonsteroidal anti-inflammatory drugs (NSAIDs), antibiotics, methotrexate, TNF inhibitors, IL-6 inhibitors, retinoids, bisphosphonates, sulfonamides, corticosteroids, and morphine-type analgesics; and food supplements and plant extracts.

In one embodiment, the kit-of-parts of the invention comprises a first part comprising at least one of compounds 001-014, or a pharmaceutically acceptable salt or solvate thereof, and a second part comprising another active ingredient, e.g., an active ingredient as described hereinabove.

### Method of Treatment

This invention also relates to the use of a compound as described above or a pharmaceutical composition thereof in the treatment of SAPHO syndrome.

This invention also relates to the use of a compound as described above or a pharmaceutical composition thereof in the manufacture of a medicament for the treatment of SAPHO syndrome.

This invention also relates to a method for the treatment of SAPHO syndrome in a subject in need thereof, comprising a step of administering to said subject a therapeutically effective amount of a compound as described above or a pharmaceutical composition thereof.

### EXAMPLES

The present invention is further illustrated by the following examples.

### Example 1: Assessment of the Efficacy of NMN on SAPHO Syndrome in Patients

### Purpose

This case report investigated the effects of nicotinamide mononucleotide derivatives according to the invention in a patient suffering from SAPHO syndrome. Especially, the effects of nicotinamide mononucleotide (NMN), i.e. compound 001, were investigated.

### Method

NMN was orally administered at a dose of 500 mg/day, once a day in the morning, for 12 months.

The patient reported that SAPHO syndrome manifested with osteitis in the upper right chest area managed with NSAIDs. At the start of the treatment, the patient reported strong pain and swelling (7.5/10) in joints affected by the disease. The patient also suffered from fatigue.

In the absence of SAPHO syndrome specific questionnaire, efficacy of the treatment was assessed using self-assessment questionnaires: the Bath Ankylosing Spondylitis Disease Activity Index (BASDAI) which is the gold standard for measuring and following disease activity in Ankylosing Spondylitis, and the Bath Ankylosing Spondylitis Functional Index (BAFSI) for measuring and monitoring functional ability in patients with ankylosing spondylitis. Scores of 4 or greater suggest suboptimal control of disease. Evaluation was performed before start of the treatment, and after 12 months of treatment.

### Results

The scores recorded for BASDAI and BASFI are reported below, before vs after treatment with NMN.

### BASDAI:

before NMN: 5.4
   At the start of the treatment, the highest score reported pain and swelling (score 7.5/10) in joints other than neck back and hip. Overall level of fatigue was graded at 6.5/10. Discomfort in the areas affected by the disease was graded at 6.5/10.
after NMN: 3
   Following 12 months of administration, the treatment significantly improved fatigue, pain and swelling of the joints and discomfort.

### BASFI:

Overall, BAFSI results at the start of the experiment reported a low level of functional impairment. However, small improvements were observed:
before NMN: 2.4
   Before treatment, the patient reported having difficulties with reaching up high shelf (4/10), and expressed having difficulties with physically demanding activities (7/10).
after NMN: 2.1
   Following one year treatment, the patient reported improvement in performing physically demanding activities (score 4.5/10).

### Conclusion

The BASDAI and BASFI questionnaires used in this case report to evaluate the efficacy of the treatment gave insight into the quality of life and functional improvements provided by the treatment.

A patient suffering from SAPHO syndrome, especially presenting upper right chest osteitis and fatigue, experienced a reduction of the symptoms of SAPHO syndrome with a nicotinamide mononucleotide compound, most notably a significant improvement of pain of swelling in joints affected by the disease and overall improvement of fatigue.

### Example 2: Synthesis of Nicotinamide Mononucleotide Derivatives

### Materials and Methods

All materials were obtained from commercial suppliers and used without further purification. Thin-layer chromatography was performed on TLC plastic sheets of silica gel 60F254 (layer thickness 0.2 mm) from Merck. Column chromatography purification was carried out on silica gel 60 (70-230 mesh ASTM, Merck). Melting points were determined either on a digital melting point apparatus (Electrothermal IA 8103) and are uncorrected or on a Kofler bench type WME (Wagner & Munz). IR, ¹H, ¹⁹F and ¹³C NMR spectra confirmed the structures of all compounds. IR spectra were recorded on a Perkin Elmer Spectrum 100 FT-IR spectrometer and NMR spectra were recorded, using CDCl₃, CD₃CN, D₂O or DMSO-d₆ as solvent, on a Bruker AC 300, Advance DRX 400 and Advance DRX 500 spectrometers, for ¹H, 75 or 100 MHz for ¹³C and 282 or 377 MHz for ¹⁹F spectra. Chemical shifts (δ) were expressed in parts per million relative to the signal indirectly (i) to CHCl₃ (δ 7.27) for ¹H and (ii) to CDCl₃ (δ 77.2) for ¹³C and directly (iii) to CFCl₃ (internal standard) (δ 0) for ¹⁹F. Chemical shifts are given in ppm and peak multiplicities are designated as follows: s, singlet; br s, broad singlet; d, doublet; dd, doublet of doublet; t, triplet; q, quadruplet; quint, quintuplet; m, multiplet. The high-resolution mass spectra (HRMS) were recorded on a Waters spectrometer using electrospray-TOF ionization (ESI-TOF).

### Step 1: Synthesis of Compounds of Formula (A-1-i)

The compound of Formula (D) (1.0 equiv.) is dissolved in dichloromethane. Nicotinamide of Formula (E) (1.50 equiv.) and TMSOTf (1.55 equiv.) are added at room temperature. The reaction mixture is heated under reflux and stirred until the reaction is complete. The mixture is cooled to room temperature and filtered. The filtrate is concentrated to dryness to give tetraacetate (A-1-i).

### Step 2: Synthesis of the Compound 005

Tetraacetate (A-1-i) is dissolved in methanol and cooled to −10° C. Ammonia 4.6 M in methanol (3.0 equivalents) at −10° C. is added and the mixture is stirred at this temperature until the reaction is complete. Dowex HCR (H+) resin is added up to pH 6-7. The reaction mixture is heated to 0° C. and filtered. The resin is washed with a mixture of methanol and acetonitrile. The filtrate is concentrated to dryness. The residue is dissolved in the acetonitrile and concentrated to dryness. The residue is dissolved in the acetonitrile to give a solution of the compound 005.

### Step 3: Synthesis of the Compound of Formula (B-i)

The solution of the crude compound 005 in acetonitrile is diluted with trimethyl phosphate (10.0 equivalents). The acetonitrile is distilled under vacuum and the mixture is cooled to −10° C. Phosphorus oxychloride (4.0 equivalents) is added at 10° C. and the mixture is stirred at 10° C. until the reaction is complete.

### Steps 4 and 5: Synthesis of the Compounds 001 and 009

The mixture obtained in step 3 above is hydrolyzed by the addition of a 50/50 mixture of acetonitrile and water, followed by the addition of methyl tert-butyl ether. The mixture is filtered and the solid is dissolved in water. The aqueous solution is neutralized by the addition of sodium bicarbonate and extracted with dichloromethane. The aqueous layer is concentrated to dryness to give a crude mixture of NMN (compound 001) and di-NMN (compound 009).

Compounds 001 and 009 are separated by purification on Dowex 50w×8 with water elution. The fractions comprising compound 001 are concentrated and further purified by a silica gel chromatographic column. The fractions containing compound 009 are concentrated to dryness. The residue is purified by column chromatography on silica gel (gradient isopropanol/water). Pure fractions are combined and concentrated. The residue is freeze-dried to afford compound 009 as a beige solid.

Characterization of compound 009: ³¹P RMN: δ (ppm, reference 85% H₃PO₄: 0 ppm in D₂O)=−11.72; ¹H RMN: δ (ppm, reference TMS: 0 ppm in D₂O)=4.20 (ddd, J_{H-H}=11.9, 3.5, 2.4 Hz, 2H), 4.35 (ddd, J_{H-H}=11.9, 3.9, 2.2 Hz, 2H), 4.43 (dd, J_{H-H}=5.0, 2.6 Hz, 2H), 4.53 (t, J_{H-H}=5.0 Hz, 2H), 4.59 (m, 2H), 6.16 (d, J_{H-H}=5.4 Hz, 2H), 8.26 (dd, J_{H-H}=8.1, 6.3 Hz, 2H), 8.93 (d, J_{H-H}=8.1 Hz, 2H), 9.25 (d, J_{H-H}=6.2 Hz, 2H), 9.41 (s, 2H); ¹³C RMN: δ (ppm, reference TMS: 0 ppm in D₂O)=64.84 (CH₂), 70.73 (CH), 77.52 (CH), 87.11 (CH), 99.88 (CH), 128.65 (CH), 133.89 (Cq), 139.84 (CH), 142.54 (CH), 146.04 (CH), 165.64 (Cq); MS (ES+): m/z=122.8 [Mnicotinamide+H]+, 650.8 [M+H]+.

### Synthesis of Compound 010

Phosphorus oxychloride (3.0 eq.) is added to trimethylphosphate (20.0 eq.) at −5° C. β-NR chloride (1.0 eq.) is added by portions at −5° C. and the reaction mixture stirred overnight at −5° C. Morpholine (3.0 eq.) is added dropwise at −10/0° C. and the mixture stirred for 2-3 h. alpha-NMN (compound 002) (1.0 eq.) is then added by portions at −5° C. and the reaction mixture stirred at −5° C. overnight. Hydrolysis is performed by dropwise addition of water (5 vol.) at −10/0° C. and the mixture is stirred until complete homogenization at 10-15° C. The reaction mixture is then extracted with dichloromethane (6*10 vol.) and the aqueous phase neutralized by eluting through Purolite A600E formate form resin (theoretical amount to neutralize HCl coming from POCl₃). The eluate is then concentrated on vacuum at 45/50° C. to give the crude containing the α,β-diNMN (compound 010). Elution with water through Dowex 50w×8 100-200 mesh H⁺ form resin allows removing of some impurities. Fractions containing compound 010 are combined and concentrated on vacuum at 45-50° C. The crude is then purified by preparative chromatography on Luna Polar RP 10 μm stationary phase with elution with a 10 mM NaH₂PO₄ aqueous solution. Pure fractions are combined and eluted with water on Purolite C100EH H⁺ form resin (needed quantity to fully exchange Na⁺ by H⁺), then eluted on Purolite A600E acetate form resin (needed quantity to fully exchange H₂PO₄⁻ by acetate). The eluate is concentrated on vacuum and the residue freeze-dried to afford compound 010 as a white solid.

³¹P RMN: δ (ppm, reference 85% H₃PO₄: 0 ppm in D₂O)=−11.87, −11.69, −11.46, −11.29; ¹H RMN: δ (ppm, reference TMS: 0 ppm in D₂O)=4.10 (ddd, J=11.1, 6.1, 3.1 Hz, 1H), 4.15-4.25 (m, 2H), 4.36 (ddd, J=12.2, 4.4, 2.4 Hz, 1H), 4.40 (dd, J=4.9, 2.4 Hz, 1H), 4.44 (dd, J=5.0, 2.7 Hz, 1H), 4.53 (t, J=5.0 Hz, 1H), 4.5 (m, 1H), 4.85 (m, 1H), 4.92 (t, J=5.3 Hz, 1H), 6.15 (d, J=5.5 Hz, 1H), 6.51 (d, J=5.7 Hz, 1H), 8.14 (dd, J=8.0, 6.3 Hz, 1H), 8.26 (dd, J=8.1, 6.3 Hz, 1H), 8.88 (d, J=8.1 Hz, 1H), 8.92 (d, J=8.1 Hz, 1H), 9.02 (d, J=6.3 Hz, 1H), 9.24 (s, 1H), 9.26 (d, J=6.4 Hz, 1H), 9.40 (s, 1H); ¹³C RMN: δ (ppm, reference TMS: 0 ppm in D₂O)=64.83, 64.87 (CH₂), 65.30, 65.35 (CH₂), 70.65 (CH), 70.74 (CH), 71.92 (CH), 77.51 (CH), 87.03, 87.10 (CH), 87.19, 87.26 (CH), 96.57 (CH), 99.83 (CH), 126.89 (CH), 128.54 (CH), 132.44 (Cq), 133.81 (Cq), 139.85 (CH), 140.92 (CH), 142.50 (CH), 143.49 (CH), 145.06 (CH), 145.97 (CH), 165.64 (Cq), 165.88 (Cq); MS (ES+): m/z=122.8 [Mnicotinamide+H]+, 650.9 [M+H]+.

### Synthesis of Compound of Formula 011

Phosphorus oxychloride (3.0 eq.) is added to trimethylphosphate (20.0 eq.) at −5° C. α-NR chloride (1.0 eq.) is added by portions at −5° C. and the reaction mixture stirred overnight at −5° C. Morpholine (3.0 eq.) is added dropwise at −10/0° C. and the mixture stirred for 2-3 h. α-NMN (compound 002) (1.0 eq.) is then added by portions at −5° C. and the reaction mixture stirred at −5° C. overnight. Hydrolysis is performed by dropwise addition of water (5 vol.) at −10/0° C. and the mixture is stirred until complete homogenization at 10-15° C. The reaction mixture is then extracted with dichloromethane (6*10 vol.) and the aqueous phase neutralized by eluting through Purolite A600E formate form resin (theoretical amount to neutralize HCl coming from POCl₃). The eluate is then concentrated on vacuum at 45/50° C. to give the crude containing the α,α-diNMN (compound 011). Elution with water through Dowex 50w×8 100-200 mesh H⁺ form resin allows removing of some impurities. Fractions containing the compound 011 are combined and concentrated on vacuum at 45-50° C. The crude is then purified by preparative chromatography on Luna Polar RP 10 μm stationary phase with elution with a 10 mM NaH₂PO₄ aqueous solution. Pure fractions are combined and eluted with water on Purolite C100EH H⁺ form resin (needed quantity to fully exchange Na⁺ by H⁺), then eluted on Purolite A600E acetate form resin (needed quantity to fully exchange H₂PO₄⁻ by acetate). The eluate is concentrated on vacuum and the residue freeze-dried to afford compound 011 as a white solid.

³¹P RMN: δ (ppm, reference 85% H₃PO₄: 0 ppm in D₂O)=−11.40; ¹H RMN: δ (ppm, reference TMS: 0 ppm in D₂O)=4.14 (ddd, J=11.4, 3.4, 2.8 Hz, 2H), 4.23 (ddd, J=11.6, 3.3, 2.8 Hz, 2H), 4.44 (dd, J=4.8, 2.3 Hz, 2H), 4.88 (m, 2H), 4.96 (t, J=5.3 Hz, 2H), 6.54 (d, J=5.7 Hz, 2H), 8.15 (dd, J=8.1, 6.2 Hz, 2H), 8.89 (d, J=8.1 Hz, 2H), 9.05 (d, J=6.3 Hz, 2H), 9.26 (s, 2H); ¹³C RMN: δ (ppm, reference TMS: 0 ppm in D₂O)=65.37 (CH2), 70.70 (CH), 71.95 (CH), 87.30 (CH), 96.62 (CH), 126.91 (CH), 132.45 (Cq), 140.94 (CH), 143.52 (CH), 145.07 (CH), 165.90 (Cq); MS (ES+): m/z=122.7 [Mnicotinamide+H]+, 650.8 [M+H]+.

1-15. (canceled)

16. A method for treating SAPHO syndrome in a subject in need thereof, said method comprising administering to said subject a therapeutically effective amount of a compound of Formula (I);
wherein the subject suffers from:
one first symptom which is an osteoarticular symptom of SAPHO syndrome selected from synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes; and
at least one second symptom of SAPHO syndrome, different from the first symptom, selected from:
osteoarticular symptoms selected from synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes;
cutaneous symptoms selected from palmoplantar pustulosis, acne, hidradenitis suppurativa, and pustular psoriasis; and
symptoms selected from fatigue, fever, inflammatory bowel disease (IBD), Crohn disease, venous thrombosis, hypertrophic pachymeningitis, uveitis, sciatica, AA amyloidosis with related renal involvement, pleural abnormalities with parenchymal changes and pleural abnormalities with pleural changes;
and wherein the subject does not suffer from psoriatic arthritis; and
wherein the compound of Formula (I) is: 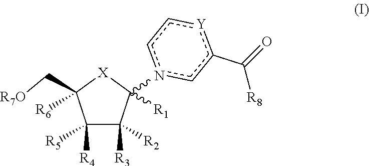
or a pharmaceutically acceptable salt or solvate thereof; wherein:
X is selected from O, CH₂, S, Se, CHF, CF₂ and C═CH₂;
R₁ is selected from H, azido, cyano, (C₁-C₈)alkyl, (C₁-C₈)thio-alkyl, (C₁-C₈)heteroalkyl and OR; wherein R is selected from H and (C₁-C₈)alkyl;
R₂, R₃, R₄ and R₅ are independently selected from H, halogen, azido, cyano, hydroxyl, (C₁-C₁₂)alkyl, (C₁-C₁₂)thio-alkyl, (C₁-C₁₂)heteroalkyl, (C₁-C₁₂)haloalkyl and OR; wherein R is selected from H, (C₁-C₁₂)alkyl, —C(O)(C₁-C₁₂)alkyl, —C(O)NH(C₁-C₁₂)alkyl, —C(O)O(C₁-C₁₂)alkyl, —C(O)aryl, —C(O)(C₁-C₁₂)alkyl-(C₅-C₁₂)aryl, —C(O)NH(C₁-C₁₂)alkyl-(C₅-C₁₂)aryl, —C(O)O(C₁-C₁₂)alkyl-(C₅-C₁₂)aryl and —C(O)CHR_{AA}NH₂; wherein R_{AA} is a side chain selected from a proteinogenic amino acid;
R₆ is selected from H, azido, cyano, (C₁-C₈)alkyl, (C₁-C₈)thio-alkyl, (C₁-C₈)heteroalkyl and OR; wherein R is selected from H and (C₁-C₈)alkyl;
R₇ is selected from H, P(O)R₉R₁₀, P(S)R₉R₁₀ and 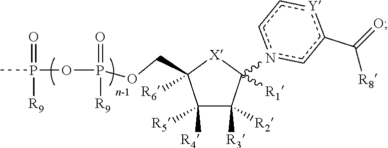
wherein:
R₉ and R₁₀ are independently selected from OH, OR₁₁, NR₁₃R₁₄, (C₁-C₈)alkyl, (C₂-C₈)alkenyl, (C₂-C₈)alkynyl, (C₃-C₁₀)cycloalkyl, (C₅-C₁₂)aryl, (C₅-C₁₂)aryl-(C₁-C₈)alkyl, (C₁-C₈)alkyl-(C₅-C₁₂)aryl, (C₁-C₈)heteroalkyl, (C₃-C₈)heterocycloalkyl, (C₅-C₁₂)heteroaryl and NHCR_{α}R_{α′}C(O)OR₁₂; wherein:
R₁₁ is selected from (C₁-C₁₀)alkyl, (C₃-C₁₀)cycloalkyl, (C₅-C₁₂)aryl, (C₁-C₁₀)alkyl-(C₅-C₁₂)aryl, substituted (C₅-C₁₂)aryl, (C₁-C₁₀)heteroalkyl, (C₁-C₁₀)haloalkyl, —(CH₂)ₘC(O)(C₁-C₁₅)alkyl, —(CH₂)ₘOC(O)(C₁-C₁₅)alkyl, —(CH₂)ₘOC(O)O(C₁-C₁₅)alkyl, —(CH₂)ₘSC(O)(C₁-C₁₅)alkyl, —(CH₂)ₘC(O)O(C₁-C₁₅)alkyl, —(CH₂)ₘC(O)O(C₁-C₁₅)alkyl-(C₅-C₁₂)aryl; wherein m is an integer selected from 1 to 8; and —P(O)(OH)OP(O)(OH)₂; and an internal or external counterion;
R₁₂ is selected from hydrogen, (C₁-C₁₀)alkyl, (C₂-C₈)alkenyl, (C₂-C₈)alkynyl, (C₁-C₁₀)haloalkyl, (C₃-C₁₀)cycloalkyl, (C₃-C₁₀)heterocycloalkyl, (C₅-C₁₂)aryl, (C₁-C₄)alkyl-(C₅-C₁₂)aryl and (C₅-C₁₂)heteroaryl; wherein said aryl or heteroaryl groups are optionally substituted by one or two groups selected from halogen, trifluoromethyl, (C₁-C₆)alkyl, (C₁-C₆)alkoxy and cyano;
R₁₃ and R₁₄ are independently selected from H, (C₁-C₈)alkyl and (C₁-C₈)alkyl-(C₅-C₁₂d)aryl; and
R_{α} and R_{α′} are independently selected from an hydrogen, (C₁-C₁₀)alkyl, (C₂-C₁₀)alkenyl, (C₂-C₁₀)alkynyl, (C₃-C₁₀)cycloalkyl, (C₁-C₁₀)thio-alkyl, (C₁-C₁₀)hydroxyalkyl, (C₁-C₁₀)alkyl-(C₅-C₁₂)aryl, (C₅-C₁₂)aryl, —(CH₂)₃NHC(═NH)NH₂, (1H-indol-3-yl)methyl, (1H-imidazol-4-yl)methyl and a side chain selected from a proteinogenic or non-proteinogenic amino acid; wherein said aryl groups are optionally substituted with a group selected from hydroxyl, (C₁-C₁₀)alkyl, (C₁-C₆)alkoxy, halogen, nitro and cyano; or
R₉ and R₁₀ together with the phosphorus atom to which they are attached form a 6-membered ring wherein —R₉-R₁₀— represents —O—CH₂—CH₂—CHR—O—; wherein R is selected from hydrogen, (C₅-C₆)aryl and (C₅-C₆)heteroaryl; wherein said aryl or heteroaryl groups are optionally substituted by one or two groups selected from halogen, trifluoromethyl, (C₁-C₆)alkyl, (C₁-C₆)alkoxy and cyano;
X′ is selected from O, CH₂, S, Se, CHF, CF₂ and C═CH₂;
R_{1′} is selected from H, azido, cyano, (C₁-C₈)alkyl, (C₁-C₈)thio-alkyl, (C₁-C₈)heteroalkyl and OR; wherein R is selected from H and (C₁-C₈)alkyl;
R_{2′}, R_{3′}, R_{4′} et R_{5′} are independently selected from H, halogen, azido, cyano, hydroxyl, (C₁-C₁₂)alkyl, (C₁-C₁₂)thio-alkyl, (C₁-C₁₂)heteroalkyl, (C₁-C₁₂)haloalkyl and OR; wherein R is selected from H, (C₁-C₁₂)alkyl, —C(O)(C₁-C₁₂)alkyl, —C(O)NH(C₁-C₁₂)alkyl, —C(O)O(C₁-C₁₂)alkyl, —C(O)aryl, —C(O)(C₁-C₁₂)alkyl-(C₅-C₁₂)aryl, —C(O)NH(C₁-C₁₂)alkyl-(C₅-C₁₂)aryl, —C(O)O(C₁-C₁₂)alkyl-(C₅-C₁₂)aryl and —C(O)CHR_{AA}NH₂; wherein Ra is a side chain selected from a proteinogenic amino acid;
R_{6′} is selected from H, azido, cyano, (C₁-C₈)alkyl, (C₁-C₈)thio-alkyl, (C₁-C₈)heteroalkyl and OR; wherein R is selected from H and (C₁-C₈)alkyl;
R_{8′} is selected from H, OR, NR₁₅R_{16′}, NH—NHR_{15′}, SH, CN, N₃ and halogen; wherein R is selected from H and (C₁-C₈)alkyl, and R_{15′} and R_{16′} are independently selected from H, (C₁-C₈)alkyl and (C₁-C₈)alkyl-(C₅-C₁₂)aryl and —CHR_{AA}CO₂H wherein R_{AA} is a side chain selected from a proteinogenic or non-proteinogenic amino acid;
Y′ is selected from CH, CH₂, CHCH₃, C(CH₃)₂ and CCH₃;
n is an integer selected from 1 to 3; [symbol]
represents the point of attachment; [symbol]
represents a single or double bond depending on Y′; and [symbol]
represents the alpha or beta anomer depending on the position of R_{1′};
R₈ is selected from H, OR, NR₁₅R₁₆, NH—NHR₁₅, SH, CN, N₃ and halogen; wherein R is selected from H and (C₁-C₈)alkyl, and R₁₅ and R₁₆ are independently selected from H, (C₁-C₈)alkyl, (C₁-C₈)alkyl-aryl and —CHR_{AA}CO₂H wherein R_{AA} is a side chain selected from a proteinogenic or non-proteinogenic amino acid;
Y is selected from CH, CH₂, CHCH₃, C(CH₃)₂ and CCH₃; [symbol]
represents a single or double bond depending on Y; and [symbol]
represents the alpha or beta anomer depending on the position of R₁.

17. The method according to claim 16, wherein X represents an oxygen.

18. The method according to claim 16, wherein R₁ and R₆ are identical and represent hydrogen.

19. The method according to claim 16, wherein R₃ and R₄ are identical and represent hydrogen.

20. The method according to claim 16, wherein R₂ and R₅ are identical and represent OH.

21. The method according to claim 16, wherein Y is selected from CH and CH₂.

22. The method according to claim 16, wherein R₇ is selected from H, P(O)R₉R₁₀ and 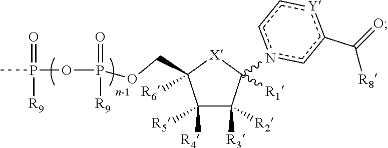
wherein
R₉ and R₁₀ are as described in claim 1;
X′ is an oxygen;
R_{1′} and R_{6′} each represents a hydrogen;
R_{2′}, R_{3′}, R_{4′} and R_{5′} are independently selected from hydrogen and OH;
R_{8′} is NH₂;
Y′ is selected from CH and CH₂;
n is equal to 2; [symbol]
represents the point of attachment; [symbol]
represents a single or double bond depending on Y′; and [symbol]
represents the alpha or beta anomer depending on the position of R_{1′}.

23. The method according to claim 16, wherein R₈ is NH₂.

24. The method according to claim 16, wherein the compound of Formula (I) is selected from:

| Compounds | |
|---|---|
| (anomers) | Structure |
| 001 (beta) | 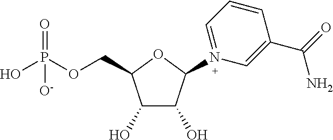 |
| 002 (alpha) | 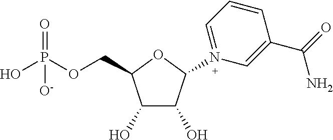 |
| 003 (beta) | 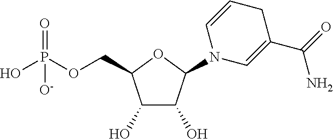 [SMILES: NC(=O)C1=CN([C@@H]2O[C@H](COP(=O)([O-])O)[C@@H](O)[C@H]2O)C=CC1] |
| 004 (alpha) | 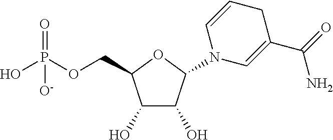 [SMILES: NC(=O)C1=CN([C@H]2O[C@H](COP(=O)([O-])O)[C@@H](O)[C@H]2O)C=CC1] |
| 005 (beta) | 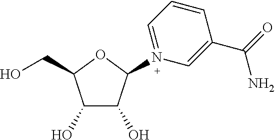 |
| 006 (alpha) | 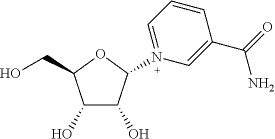 |
| 007 (beta) | 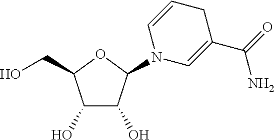 [SMILES: NC(=O)C1=CN([C@@H]2O[C@H](CO)[C@@H](O)[C@H]2O)C=CC1] |
| 008 (alpha) | 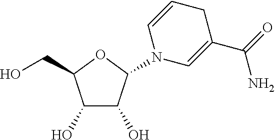 [SMILES: NC(=O)C1=CN([C@H]2O[C@H](CO)[C@@H](O)[C@H]2O)C=CC1] |
| 009 (beta, beta) | 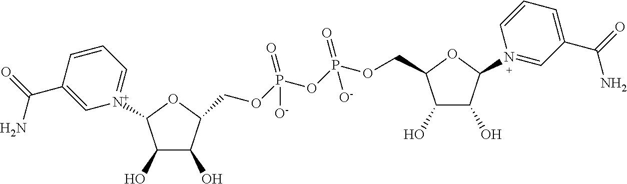 |
| 010 (beta, alpha) | 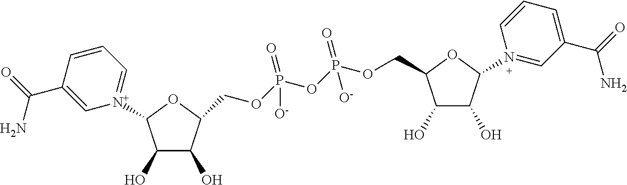 |
| 011 (alpha, alpha) | 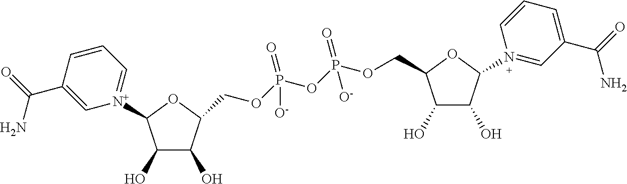 |
| 012 (beta, beta) | 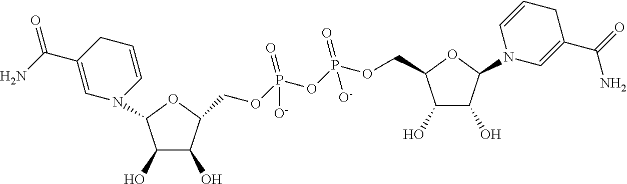 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| 013 (beta, alpha) | 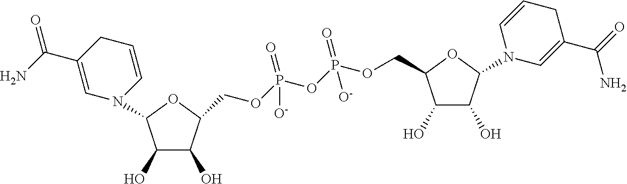 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| 014 (alpha, alpha) | 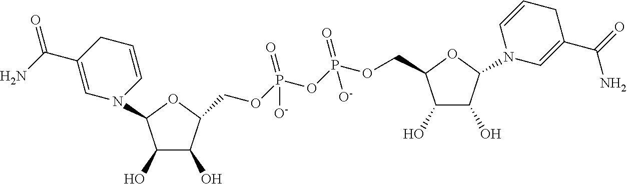 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |

and pharmaceutically acceptable salts and solvates thereof.

25. The method according to claim 16, wherein the subject suffers from two or more symptoms of SAPHO syndrome selected from synovitis, osteitis, primitive inflammatory osteitis, hyperostosis, axial spondyloarthritis, arthritis, enthesitis, diffuse idiopathic skeletal hyperostosis (DISH)-like non-marginal enthesophytes, palmoplantar pustulosis, acne, hidradenitis suppurativa, and pustular psoriasis.

26. The method according to claim 16, wherein the subject suffers from:
one or more osteoarticular symptoms of SAPHO syndrome selected from synovitis, osteitis, primitive inflammatory osteitis, hyperostosis, axial spondyloarthritis, arthritis, enthesitis, and diffuse idiopathic skeletal hyperostosis (DISH)-like non-marginal enthesophytes; and
one or more cutaneous symptoms of SAPHO syndrome selected from palmoplantar pustulosis, acne, hidradenitis suppurativa, and pustular psoriasis.

27. The method according to claim 16, wherein at least two symptoms of SAPHO syndrome are treated.

28. The method according to claim 27, wherein the at least two treated symptoms are selected from synovitis, osteitis, primitive inflammatory osteitis, hyperostosis, axial spondyloarthritis, primitive inflammatory osteitis, enthesitis, diffuse idiopathic skeletal hyperostosis (DISH)-like non-marginal enthesophytes, palmoplantar pustulosis, acne, hidradenitis suppurativa, and pustular psoriasis.

29. The method according to claim 16, wherein the compound of Formula (I) is to be administered simultaneously, separately or sequentially with at least one further pharmaceutically active agent selected from naproxen, ibuprofen, indomethacin, diclofenac, celecoxib, etoricoxib, mefenamic acid, high dose aspirin, doxycycline, minocycline, trimethoprim, sulfamethoxazole, azithromycin, clindamycin, methotrexate, etanercept, adalimumab, infliximab, certolizumab pegol, golimumab, tocilizumab, sarilumab, siltuximab, olokizumab, elsilimomab, clazakizumab, sirukumab, levilimab, acitretin, isotretinoin, retinol, retinoic acid, adapalene, alitretinoin, bexarotene, adapalene, pamidronate, risedronate, alendronate, ibandronate, zoledronic acid, etidronate, sulfasalazine, sulfomethoxasol, sulfisoxasol, colchicine, prednisone, hydrocortisone, prednisolone, dexamethasone, methylprednisolone, triamcinolone, betamethasone, oxycodone, hydrocodone, codeine, fentanyl, hydromorphone, oxymorphone, and caltonin; and/or with at least one further food supplement or plant extract selected from S-adenosylmethionine, Boswellic acids, capsaicin or Capsicum frutescens, curcumin, turmeric, avocado soybean unsaponifiables, Uncaria tomentosa, fish oil, omega 3 fatty acids (EPA and DHA), gamma linoleic acid, ginger, Zingiber officinale, cannabidiol, CBD, chondroitin, glucosamine, and harpagophytum.

30. The method according to claim 16, wherein the compound of Formula (I) is comprised within a pharmaceutical composition comprising at least one pharmaceutically acceptable carrier.
